(19) United States
(12) Patent Application Publication
(10) Pub. No.: US 2022/0117972 A1
(43) Pub. Date: Apr. 21, 2022

(54) USE OF PPAR-DELTA AGONISTS IN THE TREATMENT OF MITOCHONDRIAL MYOPATHY

(71) Applicant: Reneo Pharmaceuticals, Inc., San Diego, CA (US)

(72) Inventors: Colin O'CARROLL, San Diego, CA (US); Niall O'DONNELL, San Diego, CA (US); Lynn PURKINS, San Diego, CA (US); Alex DORENBAUM, San Diego, CA (US)

(21) Appl. No.: 17/428,090

(22) PCT Filed: Feb. 20, 2020
(86) PCT No.: PCT/US2020/019059

§ 371 Date: Aug. 3, 2021

Related U.S. Application Data
(60) Provisional application No. 62/808,137, filed on Feb. 20, 2019.

(51) Int. Cl. A61K 31/5375; A61K 31/415; A61K 31/192; A61K 31/122; A61K 31/09; A61K 31/455; A61K 31/525; A61K 31/155; A61K 31/205; A61K 31/4188; A61K 31/51; A61K 31/197; A61K 31/385; A61P 21/00

(52) U.S. Cl. CPC A61K 31/5375; A61K 31/415; A61K 31/192; A61K 31/122; A61K 31/09; A61K 31/455; A61P 21/00; A61K 31/155; A61K 31/205; A61K 31/4188; A61K 31/51; A61K 31/197; A61K 31/385; A61K 31/525

(57) ABSTRACT

Described herein is the use of PPARδ agonists in the treatment of mitochondrial myopathy. In one aspect, described herein are methods for treating a primary mitochondrial myopathy (PMM) in a mammal comprising administering to the mammal with a primary mitochondrial myopathy a peroxisome proliferator-activated receptor delta (PPARδ) agonist compound. In another aspect, described herein is a method of modulating PPARδ in a mammal with primary mitochondrial myopathy comprising administering to the mammal with primary mitochondrial myopathy PPARδ agonist compound.

### CROSS-REFERENCE

This application claims the benefit of U.S. Provisional Patent Application No. 62/808,137 filed on Feb. 20, 2019, which is incorporated herein by reference in its entirety.

### FIELD OF THE INVENTION

Described herein are methods of using a peroxisome proliferator-activated receptor delta (PPARδ) agonist in the treatment or prevention of mitochondrial myopathy.

### BACKGROUND OF THE INVENTION

Healthy mitochondria are vital to normal cellular activities. Mitochondrial dysfunction drives the pathogenesis of a wide variety of medical disorders, including acute conditions and chronic diseases. Distinct aspects of mitochondrial function, for example, bioenergetics, dynamics, and cellular signaling are well described and impairments in these activities likely contribute to disease pathogenesis. Impairments of mitochondrial function result in a family of disorders termed primary mitochondrial myopathy. Primary mitochondrial myopathies (PMM) are genetically defined disorders leading to defects of oxidative phosphorylation affecting predominantly, but not exclusively, skeletal muscle. PARδ, a member of the nuclear regulatory superfamily of ligand-activating transcriptional regulators, is expressed throughout the body. PPARδ agonists induce genes related to fatty acid oxidation and mitochondrial biogenesis. PPARδ also has anti-inflammatory properties.

### SUMMARY OF THE INVENTION

In one aspect, described herein are methods for treating a primary mitochondrial myopathy (PMM) in a mammal comprising administering to the mammal with a primary mitochondrial myopathy a peroxisome proliferator-activated receptor delta (PPARδ) agonist compound.

In another aspect, described herein is a method of modulating PPARδ in a mammal with primary mitochondrial myopathy comprising administering to the mammal with primary mitochondrial myopathy PPARδ agonist compound.

In some embodiments, treating the primary mitochondrial myopathy comprises increasing oxidative phosphorylation (OXPHOS) in the mammal, improving the mammal's exercise tolerance, decreasing pain, decreasing fatigue, improving cognition, improving overall well-being, increasing survival or a combination thereof. In some embodiments, the PPARδ agonist compound is administered to the mammal in an amount sufficient for increasing OXPHOS capacities in the mammal, up-regulating gene expression of any one of the enzymes or proteins involved in OXPHOS, or a combination thereof.

In some embodiments, the PPARδ agonist compound is administered to the mammal in an amount sufficient to improve oxidative phosphorylation capacities in the mammal, up-regulating gene expression of any one of the enzymes or proteins involved in oxidative phosporylation, or a combination thereof.

In yet another aspect, described herein is a method for increasing fatty acid oxidation (FAO) in a mammal with primary mitochondrial myopathy comprising administering to the mammal with primary mitochondrial myopathy a PPARδ agonist compound. In some embodiments, the PPARδ agonist compound is administered to the mammal in an amount sufficient to improve FAO capacities in the mammal, up-regulating gene expression of any one of the enzymes or proteins involved in FAO, or a combination thereof.

In some embodiments, the mammal with a primary mitochondrial myopathy has: at least one mutation or deletion in at least one mitochondrial DNA (mtDNA) gene; at least one mitochondrial DNA (mtDNA) defect; at least one mutation or deletion in at least one nuclear DNA (nDNA) gene involved in mitochondrial function; or a combination thereof.

In some embodiments, the at least one mutation in at least one mitochondrial DNA (mtDNA) gene comprises a mutation selected from m.3243A>G, m.8344A>G, m.8993T>G, m.13513G>A, m.11778G>A, m.14484T>C, and a combination thereof. In some embodiments, the at least one mutation in at least one mitochondrial DNA (mtDNA) gene comprises mutation m.3243A>G.

In some embodiments, the at least one mutation in at least one mitochondrial DNA (mtDNA) gene comprises a mutation selected from a 8284 bp deletion, a 6277 bp deletion, a 4977 bp deletion, and a combination thereof.

In some embodiments, the at least one mutation or deletion in at least one nuclear DNA (nDNA) gene involved in mitochondrial function comprises at least one mutation or deletion in a nDNA gene encoding complex I (NADH:ubiquinone oxidoreductase), complex II (succinate dehydrogenase), complex III (CoQ-cytochrome c reductase), complex IV (cytochrome c oxidase), complex V (ATP synthase), an aminoacyl-tRNA synthetase, a release factor, an elongation factor, a mitoribosomal protein, solute carriers of thiamine and phosphate, or a combination thereof. In some embodiments, the gene encoding the complex I comprises NDUFS1, NDUFS2, NDUFS3, NDUFS4, NDUFS6, NDUFS7, NDUFS8, NDUFVJ, NDUFV2, NDUFA1, NDUFA2, NDUFA9, NDUFA10, NDUFA11, NDUFA12, NDUFA13, NDUFAF2, NDUFAF6, or NDUFB11. In some embodiments, the gene encoding the complex II comprises SDHA, SDHB, SDHC, SDHD, or SDHAF1. In some embodiments, the gene encoding the complex III comprises UQCRB, BCS1L, UQCRQ, UQCRC2, CYC1, TTC19, LYRM7, UQCC2, or UQCC3. In some embodiments, the gene encoding the complex IV comprises COA5, SURF1, COX10, COX14, COX15, COX20, COX6B1, FASTKD2, SCO1, SCO2, LRPPRC, TACO1, or PET100. In some embodiments, the gene encoding the complex V comprises ATPAF2, TMEM70, ATP5E, or ATP5A1. In some embodiments, the gene encoding the aminoacyl-tRNA synthetase comprises AARS2, DARS2, EARS2, RARS2, YARS2, FARS2, HARS2, LARS2, VARS2, TARS2, IARS2, CARS2, PARS2, NARS2, KARS, GARS, SARS2, or MARS2. In some embodiments, the gene encoding the release factor comprises C12orf65. In some embodiments, the gene encoding the elongation factor comprises TUFM, TSFM, or GFM1. In some embodiments, the gene encoding the mitoribosomal protein comprises MRPS16, MRPS22, MRPL3, MRP12, or MRPL44. In some embodiments, the gene encoding the solute carriers of thiamine and phosphate comprises SLC19A3, SLC25A3, or SLC25A19.

In some embodiments, the at least one mutation or deletion in at least one nuclear DNA (nDNA) gene involved in mitochondrial function comprises at least one mutation or deletion in a nDNA gene involved in phospholipid metabolism, metabolism of toxic compounds, disulfide relay system, iron-sulfur protein assembly, tRNA modification, mRNA processing, mitochondrial fusion or fission, deoxynucleotide triphosphate synthesis, protein quality control and degradation, ATP and ADP transport, or a combination thereof. In some embodiments, the gene involved in the phospholipid metabolism comprises AGK, SERAC1, or TAZ. In some embodiments, the gene involved in the metabolism of toxic compounds comprises HIBCH, ECHS1, ETHE1, or MPV17. In some embodiments, the gene involved in the disulfide relay system comprises GFER. In some embodiments, the gene involved in the iron-sulfur protein assembly comprises ISCU, BOLA3, NFU1, or IBA57. In some embodiments, the gene involved in the tRNA modification comprises MTO1, GTP3BP, TRMU, PUS1, MTFMT, TRIT1, TRNT1, or TRMT5. In some embodiments, the gene involved in the mRNA processing comprises LRPPRC, TACO1, ELAC2, PNPT1, HSD17B10, MTPAP, or PTCD1. In some embodiments, the gene involved in the mitochondrial fusion and fission comprises OPA1 or MFN2. In some embodiments, the gene involved in the deoxynucleotide triphosphate synthesis comprises DGUOK, TK2, TYMP, MGME1, SUCLG1, SUCLA2, RNASEH1, C10orf2, POLG, POLG2, DNA2, or RRM2B. In some embodiments, the gene involved in the protein quality control and degradation comprises FBXL4, AFG3L2, or SPG7. In some embodiments, the gene involved the ATP and ADP transport comprises ANT1.

In some embodiments, the mammal has been diagnosed with Kearns-Sayre syndrome (KSS), Leigh syndrome, maternally inherited Leigh syndrome (MILS), Mitochondrial DNA depletion syndrome (MDS), Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy ataxia and retinitis pigmentosa (NARP), Pearson syndrome, or Progressive external ophthalmoplegia (PEO).

In some embodiments, the mammal with a primary mitochondrial myopathy also comprises a secondary mitochondrial myopathy. In some embodiments, the secondary mitochondrial myopathy is an inherited secondary mitochondrial myopathy. In some embodiments, the secondary mitochondrial myopathy is an acquired secondary mitochondrial myopathy. In some embodiments, the secondary mitochondrial myopathy involves secondary defects in OXPHOS function due to primary FAO deficiencies, or the secondary mitochondrial myopathy results from a primary OXPHOS deficiency that results in secondary FAO disease.

In some embodiments, the PPARδ agonist activates PPARδ. In some embodiments, the PPARδ agonist increases activity of PPARδ. In some embodiments, the PPARδ agonist increases mitochondrial biogenesis. In some embodiments, the PPARδ agonist increases expression or activity of a gene or protein involved in mitochondrial biogenesis. In some embodiments, the protein is peroxisome proliferator-activated receptor gamma coactivator 1-alpha (PGC-1a). In some embodiments, the PPARδ agonist increases expression or activity of a gene or protein thereof involved in oxidative phosphorylation.

In some embodiments, the PPARδ agonist increases the percentage of non-mutated mitochondrial DNA (mtDNA) relative to the proportion of mutated mtDNA. In some embodiments, the percentage of non-mutated mtDNA is increased to by at least 10% after treatment with the PPARδ agonist compound. In some embodiments, the percentage of non-mutated mtDNA is increased to by about 10% to about 20%, by about 10% to about 30%, by about 10% to about 40%, by about 10% to about 50%, by about 10% to about 60%, by about 10% to about 70%, by about 10% to about 80%, or by about 10% to about 90%, after treatment with the PPARδ agonist compound.

In some embodiments, the PPARδ agonist compound binds to and activates the cellular PPARδ and does not substantially activate the cellular peroxisome proliferator activated receptors alpha (PPARα) and gamma (PPARγ).

In some embodiments, the PPARδ agonist compound is a phenoxyalkylcarboxylic acid compound. In some embodiments, the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound. In some embodiments, the PPARδ agonist compound is a phenoxyethanoic acid compound or a phenoxyhexanoic acid compound. In some embodiments, the PPARδ agonist compound is an allyloxyphenoxyethanoic acid compound.

In some embodiments, the PPARδ agonist compound is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3,3-Bis-(4-bromo-phenyl)-allyloxy]-2-methyl-phenoxy}-acetic acid; or a pharmaceutically acceptable salt thereof.

In some embodiments, the PPARδ agonist compound is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; {4-[3,3-Bis-(4-bromo-phenyl)-allyloxy]-2-methyl-phenoxy}-acetic acid; 2-[2-methyl-4-[[3-methyl-4-[[4-(trifluoromethyl)phenyl]methoxy]phenyl]thio]phenoxy]-acetic acid; (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); {2-methyl-4-[5-methyl-2-(4-trifluoromethyl-phenyl)-2H-[1,2,3]triazol-4-ylmethylsylfanyl]-phenoxy}-acetic acid; (R)-3-methyl-6-(2-((5-methyl-2-(4-(trifluoromethyl)phenyl)-1H-imidazol-1-yl)methyl)phenoxy)hexanoic acid, (R)-3-methyl-6-(2-((5-methyl-2-(6-(trifluoromethyl)pyridin-3-yl)-1H-imidazol-1-yl)methyl)phenoxy)hexanoic acid; 2-(2-methyl-4-(((2-(4-(trifluoromethyl)phenyl)-2H-1,2,3-triazol-4-yl)methyl)thio)phenoxy)acetic acid; and (R)-2-(4-((2-ethoxy-3-(4-(trifluoromethyl)phenoxy)propyl)thio)phenoxy)acetic acid; or a pharmaceutically acceptable salt thereof.

In some embodiments, the PPARδ agonist compound is a compound selected from the group consisting of PPARδ agonist is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; or a pharmaceutically acceptable salt thereof.

In some embodiments, the PPARS agonist compound is (E)-[4-[3-(4-Fluorophenyl)-3-[4-[3-(morpholin-4-yl)propynyl]phenyl]allyloxy]-2-methyl-phenoxy]acetic acid (Compound 1), or a pharmaceutically acceptable salt thereof. In some embodiments, the PPARS agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 10 mg to about 500 mg. In some embodiments, the PPARS agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg. In some embodiments, the PPARS agonist compound is (E)-[4-[3-(4-Fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 75 mg to about 125 mg. In some embodiments, the PPARS agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg. In some embodiments, the PPARS agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 100 mg.

In another aspect, provided herein is a method for treating a primary mitochondrial myopathy in a mammal comprising administering to the mammal with a primary mitochondrial myopathy a PPARS agonist compound, wherein the PPARS agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof.

In some embodiments, treating the primary mitochondrial myopathy comprises increasing oxidative phosphorylation (OXPHOS) in the mammal, improving the mammal's exercise tolerance, improving muscle histology, improving mitochondrial DNA copy number, improving heteroplasmy levels, improving the quality of mitochondria, decreasing pain, decreasing fatigue, improving cognition, improving overall well-being, increasing survival or a combination thereof.

In some embodiments, the peroxisome proliferator-activated receptor delta (PPARδ) agonist compound is administered to the mammal in an amount sufficient for increasing OXPHOS capacities in the mammal, up-regulating gene expression of any one of the enzymes or proteins involved in OXPHOS, or a combination thereof. In some embodiments, the peroxisome proliferator-activated receptor delta (PPARδ) agonist compound is administered to the mammal in an amount sufficient for increasing fatty acid oxidation (FAO) capacities in the mammal, up-regulating gene expression of any one of the enzymes or proteins involved in FAO, or a combination thereof.

In another aspect, provided herein is a method for treating a primary mitochondrial myopathy in a human comprising administering to the mammal with a primary mitochondrial myopathy a PPARδ agonist compound, wherein after treatment the mammal has improvement in one or more of pain, cognition, physical endurance, muscle strength, feeling of well-being, or increasing survival.

In some embodiments, the improvement is physical endurance. In some embodiments, the improvement is physical endurance as demonstrated by one or more of improvement in walking endurance, or sit to stand test. In some embodiments, the improvement is muscle strength. In some embodiments, the muscle strength is measured by grip strength, or leg strength. In some embodiments, the improvement is increasing survival of the human.

In some embodiments, the PPARδ agonist compound is: (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg. In some embodiments, (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 50 mg to about 200 mg. In some embodiments, (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 75 mg to about 125 mg. In some embodiments, (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 50 mg. In some embodiments, (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 100 mg. In some embodiments, (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof is systemically administered to the mammal in the form an oral solution, oral suspension, powder, pill, tablet or capsule. In some embodiments, the PPARS agonist compound is administered to the mammal daily. In some embodiments, the PPARS agonist compound is administered to the mammal once daily.

In some embodiments, the PPARS agonist is systemically administered to the mammal. In some embodiments, the PPARS agonist is administered to the mammal orally, by injection or intravenously. In some embodiments, the PPARS agonist is administered to the mammal in the form of an oral solution, oral suspension, powder, pill, tablet or capsule.

In one aspect, described herein is a pharmaceutical composition comprising PPARS agonist and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In any of the aforementioned aspects are further embodiments in which the effective amount of the PPARS agonist (e.g. Compound 1, or a pharmaceutically acceptable salt thereof), is: (a) systemically administered to the mammal; and/or (b) administered orally to the mammal; and/or (c) intravenously administered to the mammal; and/or (d) administered by injection to the mammal; and/or (e) administered non-systemically or locally to the mammal.

In any of the aforementioned aspects are further embodiments comprising single administrations of the effective amount of the PPARS agonist (e.g. Compound 1, or a pharmaceutically acceptable salt thereof), including further embodiments in which the PPARS agonist (e.g. Compound 1, or a pharmaceutically acceptable salt thereof), is administered once daily to the mammal or is administered to the mammal multiple times over the span of one day. In some embodiments, the PPARδ agonist (e.g. Compound 1, or a pharmaceutically acceptable salt thereof), is administered on a continuous dosing schedule. In some embodiments, the PPARδ agonist is administered on a continuous daily dosing schedule.

In any of the aforementioned aspects or embodiments involving the treatment of a disease or condition are further embodiments comprising administering at least one additional agent in addition to the administration of a PPARδ agonist (e.g. Compound 1, or a pharmaceutically acceptable salt thereof). In some embodiments, the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, KH176, or a combination thereof. In some embodiments, the at least one additional therapeutic is an odd-chain fatty acid, odd-chain fatty ketone, L-carnitine, or combinations thereof. In some embodiments, the at least one additional therapeutic is triheptanoin, n-heptanoic acid, a triglyceride, or a salt or thereof, or combinations thereof.

In some embodiments, the mammal is a human.

Articles of manufacture, which include packaging material, a PPARδ agonist compound described herein (e.g. Compound 1, or a pharmaceutically acceptable salt thereof), or a pharmaceutically acceptable salt thereof, within the packaging material, and a label that indicates that the PPARδ agonist compound is used for modulating the activity of PPARδ, or for the treatment, prevention or amelioration of one or more symptoms of a mitochondrial myopathy are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows the impact of administering Compound 1 (100 mg once a day for 12 weeks) to genetically diagnosed primary mitochondrial myopathy patients (mtDNA defects) with myopathy on the 12-minute walk test. Improvements in the 12-minute walk test over the course of the 12-week treatment regimen is shown for nine patients.

FIG. 2 shows the impact of administering Compound 1 (100 mg once a day for 12 weeks) to genetically diagnosed primary mitochondrial myopathy patients (mtDNA defects) with myopathy on pain scores. The mean brief pain index (BPI) score of nine patients administered Compound 1 decreased over the course of the 12-week treatment regimen.

### DETAILED DESCRIPTION

Healthy mitochondria are vital to normal cellular activities. Mitochondria harvest energy in the form of ATP and simultaneously regulate cellular metabolism. Mitochondria perform many key roles in the cell including oxidative phosphorylation, the oxidation of fatty acids (β-oxidation), central carbon metabolism, and the biosynthesis of intermediates for cell growth.

The prime pathway for the degradation of fatty acids is mitochondrial fatty acid β-oxidation (FAO). FAO is a key metabolic pathway for energy homoeostasis in organs such as the liver, heart and skeletal muscle. Fatty acid transport proteins (FATPs) are integral membrane proteins that enhance the uptake of long chain and very long chain fatty acids into cells. In the cytosol, fatty acids are activated to acyl-coenzyme A (CoA) esters by acyl-CoA synthetases before they can be directed into several different metabolic pathways, such as lipid synthesis and FAO. FAO requires mitochondrial import of acyl-CoA. Because the mitochondrial membrane is impermeable to acyl-CoAs, the carnitine cycle is needed for import into the mitochondria. This system requires L-carnitine and is composed of two acyltransferases, carnitine palmitoyltransferases 1 and 2 (CPT1 and CPT2), and carnitine acylcarnitine translocase (CACT). Inside the mitochondrion, acyl-CoAs are degraded via 3-oxidation, which is a cyclic process of four enzymatic steps. Each cycle shortens the acyl-CoA by releasing the two carboxy-terminal carbon atoms as acetyl-CoA. FAO not only produces acetyl-CoA to fuel the Krebs cycle (also known as the tricarboxylic acid (TCA) cycle) and ketogenesis, but also reduces flavin adenine dinucleotide (to FADH2) and nicotinamide adenine dinucleotide (to NADH), and these reduced products directly feed into the electron transport chain (respiratory chain). To be able to fully degrade fatty acids, the 0-oxidation machinery harbors different chain length-specific enzymes.

Oxidative phosphorylation (OXPHOS) is a metabolic pathway responsible for the generation of the majority of cellular energy in the form of ATP. The OXPHOS pathway includes complexes I-IV of the respiratory chain and complex V, an ATP synthase. Complex I (NADH:coenzyme Q oxidoreductase) oxidizes NADH with the reduction of coenzyme Q10 (also known as CoQ) from its ubiquinone (CoQ; Q) form to ubiquinol (QH2), generating an electrochemical gradient across the inner mitochondrial membrane. Complex II (succinate-CoQ oxidoreductase) intricately links the Krebs cycle (also known as the tricarboxylic acid (TCA) cycle) to the respiratory chain. Complex II oxidizes succinate with the reduction of CoQ from its ubiquinone (CoQ; Q) form to ubiquinol (QH2). Complex III (ubiquinol-cytochrome c oxidoreductase) catalyzes the reduction of cytochrome c by oxidation of ubiquinol with the generation of an electrochemical gradient. Complex IV (cytochrome c oxidase) is responsible for the terminal enzymatic reaction of the respiratory chain that transfers electrons (e−) to molecular oxygen and generates an electrochemical gradient. Complex V converts transmembrane electrochemical proton (H+) gradient energy into mechanical energy, which catalyses the chemical bond energy between ADP and phosphate (P) to form ATP.

Over 1,500 proteins are required for healthy mitochondrial function of which thirteen proteins are encoded by mitochondrial DNA (mtDNA) and the rest are encoded by nuclear (nDNA). About 100 proteins are directly involved in oxidative phosphorylation and the production of ATP. Mutations in nDNA or mtDNA genes that disrupt mitochondrial function lead to devastating mitochondrial diseases known as primary mitochondrial myopathies (PMM). In patients with mtDNA mutations, inheritance and clinical presentation are further complicated by the presence of multiple mtDNA genomes in an individual cell leading to a mixture of mutated and wild-type genomes (heteroplasmy) in the same cell or tissue.

Many common mitochondrial disorders are linked to dysfunction of the OXPHOS pathway. Such dysfunctions can include deficiencies in OXPHOS complex activity and/or reductions in steady-state levels of the OXPHOS complexes resulting in diminished ATP production or combinations thereof (Nsihia-Sefaa, A, and McKenzie, M, (2016), Biosci. Rep., 36, e00313, doi:10.1042/BSR20150295). The defects leading to these disorders can be caused by: 1) gene mutations of the protein subunits that encode the OXPHOS proteins; 2) mutations of the proteins required for OXPHOS complex biogenesis; or 3) mutations of the proteins necessary for replication, transcription and translation of mtDNA (id.) The OXPHOS complexs and FAO pathways are biochemically linked because NAD and FADH2 that are produced during FAO pass their electrons to the OXPHOS complexes. Studies have shown that primary disorders in one pathway cause secondary defects in the other pathway (id.)

Because mitochondria are the main source of energy production in mammalian cells, clinical features of primary mitochondrial myopathy typically involve the tissues with the highest energy requirements. Furthermore, the presence of mtDNA in all human tissues means that dysfunction occurs in multiple organ systems. The most commonly affected organ systems are the nervous, muscular, cardiac, and endocrine systems. primary mitochondrial myopathies are usually progressive conditions which produce significant disability and, in some instances, premature death, often due to cardiac or neurological involvement such as arrhythmias or seizures. Myopathy can be the only clinical feature of a mitochondrial disease but may also be part of a component of other mitochondrial diseases or disorders.

PPARδ is the most abundant PPAR isoform in skeletal muscle and has a higher expression in oxidative type I muscle fibers compared with glycolytic type II muscle fibers. Both short-term exercise and endurance training lead to increased PPARδ expression in human and rodent skeletal muscle. Rodent studies suggest that a key feature of PPARδ activation is induction of skeletal muscle fatty acid oxidation. On activation of PPARδ in skeletal muscle in mice, the fiber composition changes toward the oxidative type I with induction of fatty acid oxidation, mitochondrial respiration, oxidative metabolism, and slow-twitch contractile apparatus. In addition to the metabolic effects of PPARδ activation, PPARδ also stimulates peroxisome proliferator-activated receptor gamma coactivator-1 alpha (PGC-1α), an effect accompanied by mitochondrial biogenesis. This type of adaptation is identical to that seen in response to physical exercise, and indeed, mice with transgenic (Tg) overexpression of PPARδ exhibit increased running endurance (Wang et al., PLoS Biol. 2:e294 (2004)).

The management of patients with mitochondrial diseases is focused on strategies to reduce morbidity and mortality and early treatment of organ-specific complications. Primary mitochondrial myopathies represent an area of significant unmet medical need; there is currently no available disease-modifying therapy for patients with primary mitochondrial myopathy.

Described herein, in some embodiments, are methods and compositions for treating primary mitochondrial myopathy in a mammal comprising administering to the mammal with a primary mitochondrial myopathy a PPARδ agonist compound. Further described herein, in some embodiments, are methods and compositions for modulating PPARδ in a mammal with primary mitochondrial myopathy comprising administering to the mammal with primary mitochondrial myopathy a PPARδ agonist compound. In some embodiments, modulating PPARδ in a mammal with primary mitochondrial myopathy leads to improvement in one or more symptoms associated with primary mitochondrial myopathy. In some embodiments, the mammal is a human.

In some embodiments, the mammal having primary mitochondrial myopathy has been diagnosed with Kearns-Sayre syndrome (KSS), Leigh syndrome, maternally inherited Leigh syndrome (MILS), Mitochondrial DNA depletion syndrome (MDS), Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy ataxia and retinitis pigmentosa (NARP), Pearson syndrome, or Progressive external ophthalmoplegia (PEO).

In some embodiments, the mammal with a primary mitochondrial myopathy also comprises a secondary mitochondrial myopathy. In some embodiments, secondary mitochondrial myopathy refers to any abnormal mitochondrial function other than that resulting from a primary mitochondrial myopathy (see, for example, D. Niyazov et al. Molecular Syndromology 2016; 7; 122-137).

In some embodiments, the secondary mitochondrial myopathy is an inherited secondary mitochondrial myopathy. In some embodiments, the secondary mitochondrial myopathy involves mutations in non-OXPHOS genes. In some embodiments, the secondary mitochondrial myopathy involves secondary defects in OXPHOS function due to primary FAO deficiencies. In some embodiments, the secondary mitochondrial myopathy results from a primary OXPHOS deficiency that results in secondary FAO disease. In some embodiments, the secondary mitochondrial myopathy is an acquired secondary mitochondrial myopathy. For example, the acquired secondary mitochondrial myopathy is a result of environmental factors that cause oxidative stress including, but not limited to, aging, inflammation, and mitotoxic drugs. In some embodiments, the mitotoxic drug comprises corticosteroids, valproic acid, phenytoin, barbiturates, propofol, volatile anesthetics, nondepolarizing muscle relaxants, local anesthetics, statins, fibrates, biguanides, glitazones, beta-blockers, amiodarone, neuroleptics, antibiotics, or chemotherapeutics. In some embodiments, the chemotherapeutic is doxorubicin or a platinum based chemotherapeutic such as cisplatin.

Described herein, in some embodiments, are methods and compositions for treating a mammal with a PPARδ agonist, wherein the PPARδ agonist activates PPARS. In some embodiments, the PPARδ agonist increases expression of PPARS. In some embodiments, the PPARδ agonist increases activity of PPARS. In some embodiments, the PPARδ agonist increases expression or activity of a gene or protein thereof involved in mitochondrial function. In some embodiments, the gene is a nuclear DNA (nDNA) gene. In some embodiments, the gene is a mitochondria DNA (mtDNA) gene.

In some embodiments, the PPARδ agonist increases expression or activity of a nDNA gene, wherein the nDNA gene includes, but not limited to, NDUFS1, NDUFS2, NDUFS3, NDUFS4, NDUFS6, NDUFS7, NDUFS8, NDUFVJ, NDUFV2, NDUFA1, NDUFA2, NDUFA9, NDUFA10, NDUFA11, NDUFA12, NDUFA13, NDUFAF2, NDUFAF6, NDUFB11, SDHA, SDHB, SDHC, SDHD, SDHAF1, UQCRB, BCS1L, UQCRQ, UQCRC2, CYC1, TTC19, LYRM7, UQCC2, UQCC3, COA5, SURF1, COX10, COX14, COX15, COX20, COX6B1, FASTKD2, SCO1, SCO2, LRPPRC, TACO1, PET100, ATPAF2, TMEM70, ATP5E, ATP5A1, AARS2, DARS2, EARS2, RARS2, YARS2, FARS2, HARS2, LARS2, VARS2, TARS2, IARS2, CARS2, PARS2, NARS2, KARS, GARS, SARS2, MARS2, C12orf65, TUFM, TSFM, GFM1, MRPS16, MRPS22, MRPL3, MRP12, MRPL44, SLC19A3, SLC25A3, SLC25A19, AGK, SERAC1, TAZ, HIBCH, ECHS1, ETHE1, MPV17, GFER, ISCU, BOLA3, NFU1, IBA57, MTO1, GTP3BP, TRMU, PUS1, MTFMT, TRIT1, TRNT1, TRHT5, LRPPRC, TACO1, ELAC2, PNPT1, HSD17B10, MTPAP, PTCD1, OPA1, MFN2, DGUOK, TK2, TYMP, MGME1, SUCLG1, SUCLA2, RNASEH1, C10orf2, POLG, POLG2, DNA2, RRM2B, FBXL4, AFG3L2, SPG7, and ANT1.

In some embodiments, the nDNA gene encodes complex I (NADH:ubiquinone oxidoreductase), complex II (succinate dehydrogenase), complex III (CoQ-cytochrome c reductase), complex IV (cytochrome c oxidase), complex V (ATP synthase), an aminoacyl-tRNA synthetase, a release factor, an elongation factor, a mitoribosomal protein, solute carriers of thiamine and phosphate, or a combination thereof. In some embodiments, the gene encoding the complex I comprises NDUFS1, NDUFS2, NDUFS3, NDUFS4, NDUFS6, NDUFS7, NDUFS8, NDUFVJ, NDUFV2, NDUFA1, NDUFA2, NDUFA9, NDUFA10, NDUFA11, NDUFA12, NDUFA13, NDUFAF2, NDUFAF6, or NDUFB11. In some embodiments, the gene encoding the complex II comprises SDHA, SDHB, SDHC, SDHD, or SDHAF1. In some embodiments, the gene encoding the complex III comprises UQCRB, BCS1L, UQCRQ, UQCRC2, CYC1, TTC19, LYRM7, UQCC2, or UQCC3. In some embodiments, the gene encoding the complex IV comprises COA5, SURF1, COX10, COX14, COX15, COX20, COX6B1, FASTKD2, SCO1, SCO2, LRPPRC, TACO1, or PET100. In some embodiments, the gene encoding the complex V comprises ATPAF2, TMEM70, ATP5E, or ATP5A1. In some embodiments, the gene encoding the aminoacyl-tRNA synthetase comprises AARS2, DARS2, EARS2, RARS2, YARS2, FARS2, HARS2, LARS2, VARS2, TARS2, IARS2, CARS2, PARS2, NARS2, KARS, GARS, SARS2, or MARS2. In some embodiments, the gene encoding the release factor comprises C12orf65. In some embodiments, the gene encoding the elongation factor comprises TUFM, TSFM, or GFM1. In some embodiments, the gene encoding the mitoribosomal protein comprises MRPS16, MRPS22, MRPL3, MRP12, or MRPL44. In some embodiments, the gene encoding the solute carriers of thiamine and phosphate comprises SLC19A3, SLC25A3, or SLC25A19.

In some embodiments, the nDNA gene is involved in phospholipid metabolism, metabolism of toxic compounds, disulfide relay system, iron-sulfur protein assembly, tRNA modification, mRNA processing, mitochondrial fusion or fission, deoxynucleotide triphosphate synthesis, protein quality control and degradation, ATP and ADP transport, or a combination thereof. In some embodiments, the gene involved in the phospholipid metabolism comprises AGK, SERAC1, or TAZ. In some embodiments, the gene involved in the metabolism of toxic compounds comprises HIBCH, ECHS1, ETHE1, or MPV17. In some embodiments, the gene involved in the disulfide relay system comprises GFER. In some embodiments, the gene involved in the iron-sulfur protein assembly comprises ISCU, BOLA3, NFU1, or IBA57. In some embodiments, the gene involved in the tRNA modification comprises MTO1, GTP3BP, TRMU, PUS1, MTFMT, TRIT1, TRNT1, or TRMT5. In some embodiments, the gene involved in the mRNA processing comprises LRPPRC, TACO1, ELAC2, PNPT1, HSD17B10, MTPAP, or PTCD1. In some embodiments, the gene involved in the mitochondrial fusion and fission comprises OPA1 or MFN2. In some embodiments, the gene involved in the deoxynucleotide triphosphate synthesis comprises DGUOK, TK2, TYMP, MGME1, SUCLG1, SUCLA2, RNASEH1, C10orf2, POLG, POLG2, DNA2, or RRM2B. In some embodiments, the gene involved in the protein quality control and degradation comprises FBXL4, AFG3L2, or SPG7. In some embodiments, the gene involved the ATP and ADP transport comprises ANT1.

Described herein, in some embodiments, are methods and compositions for treating a mammal using a PPARδ agonist, wherein the PPARδ agonist increases the expression or activity of a mitochondrial DNA (mtDNA) gene. In some embodiments, the mtDNA gene comprises at least one mutation, deletion, defect, or combination thereof. In some embodiments, the at least one mutation in at least one mitochondrial DNA (mtDNA) gene comprises a mutation selected from m.3243A>G, m.8344A>G, m.8993T>G, m.13513G>A, m.11778G>A, m.14484T>C, and a combination thereof. In some embodiments, the at least one mutation in at least one mitochondrial DNA (mtDNA) gene comprises mutation m.3243A>G. In some embodiments, the mtDNA gene comprises a mutation selected from an 8284 bp deletion, a 6277 bp deletion, a 4977 bp deletion, and a combination thereof.

In some embodiments, the PPARδ agonist increases a percentage of non-mutated mitochondrial DNA (mtDNA). In some embodiments, the PPARδ agonist increases the percentage of non-mutated mtDNA by at least or about 10%, 20%, 30%, 40%, 50%, 60%, 70%, 75%, 80%, 90%, 95%, or more than 95%. In some embodiments, the PPARδ agonist increases the percentage of non-mutated mtDNA in a range of about 10% to about 90%, about 20% to about 80%, about 30% to about 70%, or about 40% to about 60%. In some embodiments, the PPARδ agonist increases the percentage of non-mutated mtDNA such that a proportion of mtDNA in a cell is substantially non-mutated. In some embodiments, the proportion of non-mutated mtDNA to mutated mtDNA in a cell is at least or about 1.25:1, 1.5:1, 1.75:1, 2.0:1, 2.5:1, 3:1, 4:1, 5:1, 6:1, 7:1, 8:1, 9:1, 10:1, or more than 10:1.

In some embodiments, the PPARδ agonist increases mitochondrial biogenesis. In some embodiments, the PPARδ agonist increases expression or activity of a gene or protein thereof involved in mitochondrial biogenesis. In some embodiments, the PPARδ agonist increases the transcription of a gene involved in mitochondrial biogenesis. In some embodiments, the PPARδ agonist increases the translation of a protein involved in mitochondrial biogenesis. In some embodiments, the protein is a transcription factor. In some embodiments, the protein is peroxisome proliferator-activated receptor gamma coactivator 1-alpha (PGC-1α).

The PPARδ agonist described herein, in some embodiments, modulates the expression or activity of PGC-1α. In some embodiments, the PPARδ agonist increases the transcription of the proliferator-activated receptor gamma coactivator 1-alpha gene. In some embodiments, the PPARδ agonist increases the translation of PGC-1a protein. In some embodiments, the PPARδ agonist modulates post-translation modifications of PGC-1α. For example, the PPARδ agonist modulates PGC-1a phosphoryalation, acetylation, deacetylation, SUMOylation, ubiquitination, O-linked β-N-acetyl glucosamination, methylation, or a combination thereof.

In some embodiments, the PPARδ agonist reduces a rate of decrease in mitochondrial biogenesis. In another embodiment, described herein is a method of increases mitochondrial biogenesis in one or more tissues of a mammal relative to a control, wherein the increases in mitochondrial biogenesis comprises a comparison of one or more measurements of mitochondrial biogenesis in the mammal after treatment with a PPARδ agonist to a baseline measurement of mitochondrial biogenesis in the same mammal. In some embodiments, the tissues of the mammal comprise muscle tissues. In another embodiment, reducing the rate of increase in mitochondrial biogenesis in the mammal comprises a return to the mammal's baseline measurement of mitochondrial biogenesis faster than the control. In a further embodiment, increasing the rate of decrease in mitochondrial biogenesis in the mammal comprises a return to the mammal's baseline measurement of mitochondrial biogenesis following a period of time in less than 95%, or less than 90%, or less than 85%, or less than 80%, or less than 75%, or less than 70%, or less than 65%, or less than 60%, or less than 55%, or less than 50% of the time to return to baseline for a control. In another embodiment, the increase in mitochondrial biogenesis in the mammal is more than the increase in mitochondrial biogenesis relative to the control. In a further embodiment, the increase in mitochondrial biogenesis in the mammal comprises more than a 1%, more than a 2%, more than a 3%, more than a 4%, more than a 5%, more than a 6%, more than a 7%, more than an 8%, more than a 9%, more than a 10%, more than a 15%, more than a 20%, more than a 25%, more than a 30%, more than a 35%, more than a 40%, more than a 45%, or more than a 50% increase in mitochondrial biogenesis relative to the mammal's baseline measurement of mitochondrial biogenesis prior to treatment with a PPARδ agonist.

Muscle tissue is soft tissue found in most animals comprising muscle cells. Muscle cells contain protein filaments that can slide past one another and produce a contraction that changes both the length and shape of the muscle cell. Muscles function to produce force and motion. There are three types of muscles in the body: a) skeletal muscle (the muscle responsible for moving extremities and external areas of the bodies); b) cardiac muscle (the heart muscle); and c) smooth muscle (the muscle that is in the walls of arteries and bowel).

The term “muscle cell” as used herein refers to any cell that contributes to muscle tissue. Myoblasts, satellite cells, myotubes, and myofibril tissues are all included in the term “muscle cells” and may all be treated using the methods described herein. Muscle cell effects may be induced within skeletal, cardiac, and smooth muscles.

Skeletal muscle, or voluntary muscle, is generally anchored by tendons to bone and is generally used to effect skeletal movement such as locomotion or in maintaining posture. Although some control of skeletal muscle is generally maintained as an unconscious reflex (e.g., postural muscles or the diaphragm), skeletal muscles react to conscious control. Smooth muscle, or involuntary muscle, is found within the walls of organs and structures such as the esophagus, stomach, intestines, uterus, urethra, and blood vessels. Unlike skeletal muscle, smooth muscle is not under conscious control. Cardiac muscle is also an involuntary muscle but more closely resembles skeletal muscle in structure and is found only in the heart. Cardiac and skeletal muscles are striated in that they contain sarcomeres that are packed into highly regular arrangements of bundles. By contrast, the myofibrils of smooth muscle cells are not arranged in sarcomeres and therefore are not striated.

Skeletal muscle is further divided into two broad types: Type I (or “slow twitch”) and Type II (or “fast twitch”). Type I muscle fibers are dense with capillaries and are rich in mitochondria and myoglobin, which gives Type I muscle tissue a characteristic red color. Type I muscle fibers can carry more oxygen and sustain aerobic activity using fats or carbohydrates for fuel. Type I muscle fibers contract for long periods of time but with little force. Type II muscle fibers may be subdivided into three major subtypes (IIa, Ix, and IIb) that vary in both contractile speed and force generated. Type II muscle fibers contract quickly and powerfully but fatigue very rapidly, and therefore produce only short, anaerobic bursts of activity before muscle contraction becomes painful.

Mitochondrial biogenesis is measured by mitochondrial mass and volume through histological section staining using a fluorescently labeled antibody specific to the oxidative-phosphorylation complexes, such as the Anti-OxPhox Complex Vd subunit antibody from Life Technologies or using mitochondrial specific dyes in live cell staining, such as the Mito-tracker probes from Life Technologies. Mitochondrial biogenesis can also be measured by monitoring the gene expression of one or more mitochondrial biogenesis related transcription factors such as PGC1α, NRF1, or NRF2 using a technique such as QPCR.

FAO is crucial for ATP production in muscle mitochondria, particularly during exercise, by providing substrates for the respiratory chain. The sources of fatty acids differ depending on the exercise intensity, with the contribution of free fatty acids increasing with exercise intensity. Mutations in any of the enzymes involved in FAO may lead to a variety of clinical symptoms in particular during fasting and in organs with high energy needs. During infancy, patients may present with cardiac symptoms such as dilated or hypertrophic cardiomyopathy and/or arrhythmias. Alternatively, FAO defects might present as a milder, later (‘adult’) onset disease, characterized by exercise-induced myopathy and rhabdomyolysis.

The PPARs (PPAR-α, PPAR-δ, PPAR-γ) are known for their transcriptional regulation of FAO. Activation of PPARs may trigger an up-regulation of gene expression of the enzymes involved in FAO resulting in an increase in residual enzyme activity and thereby correction of FAO flux in treated cells. In a study using cultured patient muscle cells, specific agonists of PPARδ (GW 072) and to a lower extent PPARα (GW 7647) stimulated FAO in control myoblasts (Djouadi, F., et al. J. Clin. Endocrinol. Metab. 90, 1791-1797, 2005).

In vitro studies with Compound 1 have demonstrated its ability to elicit a dose-dependent increase in fatty acid oxidation in human and rat muscle cell lines. In addition, Compound 1 treatment altered the expression patterns of several well-known PPARδ regulated genes in pathways important for fatty acid metabolism (CPT1b) and mitochondrial biogenesis (PGC1α) in vivo.

In some embodiments, deficiencies in FAO capacities are measured by comparing FAO capacities of a mammal identified as having a primary mitochondrial myopathy to the FAO capacities of a mammal without a primary mitochondrial myopathy (i.e. a control). In some embodiments, described herein are methods of increasing FAO capacities in a mammal with a primary mitochondrial myopathy comprising administering a PPARδ agonist compound (e.g. Compound 1, or a pharmaceutically acceptable salt thereof) to a mammal with a primary mitochondrial myopathy. In some embodiments, described herein are methods of increasing FAO capacities in a mammal with a primary mitochondrial myopathy by about 5%, about 10%, about 15%, about 20%, about 25%, about 30%, about 35%, about 40%, about 45%, about 50%, about 55%, about 60%, about 75%, about 80%, about 95%, about 100%, or more than 100% of the levels observed for a mammal without a primary mitochondrial myopathy. In some embodiments, described herein are methods of increasing FAO capacities in a mammal with a primary mitochondrial myopathy to a level substantially similar to that observed for a mammal without a primary mitochondrial myopathy comprising administering a PPARδ agonist compound (e.g. Compound 1, or a pharmaceutically acceptable salt thereof) to a mammal with a primary mitochondrial myopathy. In some embodiments, described herein are methods of restoring (i.e. normalizing by improving or increasing) FAO capacities in a mammal with a primary mitochondrial myopathy to a level substantially similar to that observed for a mammal without a primary mitochondrial myopathy comprising administering a PPARδ agonist compound (e.g. Compound 1, or a pharmaceutically acceptable salt thereof) to a mammal with a primary mitochondrial myopathy.

In some embodiments, administration of a PPARδ agonist compound (e.g. Compound 1, or a pharmaceutically acceptable salt thereof), to a mammal with a primary mitochondrial myopathy restores (i.e. normalizes by increasing) a deficiency in the activity of one or more enzymes of proteins involved in the fatty acid β-oxidation pathway. In some embodiments, restoring activity comprises increasing the activity to substantially similar levels observed in a mammal without a primary mitochondrial myopathy.

In some aspects, PPARδ agonist compound is administered in a therapeutically effective amount to a subject (e.g., a human). As used herein, the term “effective amount” or “therapeutically effective amount” refers to an amount of an active ingredient that elicits the desired biological or medicinal response, for example, reduction or alleviation of the symptoms of the condition being treated. In some embodiments of the invention, the amount of PPARδ agonist compound administered varies depending on various factors, including, but not limited to, the weight of the subject, the nature and/or extent of the subject's condition, etc.

### Compounds

A PPARδ agonist compound is a fatty acid, lipid, protein, peptide, small molecule, or other chemical entity that binds to the cellular PPARδ and, without being bound to any particular theory, elicits a downstream response, namely gene transcription, either native gene transcription or a reporter construct gene transcription, comparable to endogenous ligands such as retinoic acid or comparable to a standard reference PPARδ agonist compound such as carbacyclin.

In an embodiment, a PPARδ agonist compound is a selective agonist. As used herein, a selective PPARδ agonist compound is viewed as a chemical entity that binds to and activates the cellular PPARδ and does not substantially activate the cellular peroxisome proliferator activated receptors alpha (PPARα) and gamma (PPARγ). As used herein, a selective PPARδ agonist compound is a chemical entity that has at least a 10-fold maximum activation (as compared to endogenous receptor ligand) with a greater than 100-fold potency for activation of PPARδ relative to either or both of PPARα and PPARγ. In a further embodiment, a selective PPARδ agonist compound is a chemical entity that binds to and activates the cellular human PPARδ and does not substantially activate either or both of human PPARα and PPARγ. In a further embodiment, a selective PPARδ agonist compound is a chemical entity that has at least about a 10-fold, or about a 20-fold, or about a 30-fold, or about a 40-fold, or about a 50-fold, or about a 100-fold potency for activation of PPARδ relative to either or both of PPARα and PPARγ.

In some embodiments, a selective PPARδ agonist compound contemplated herein is capable of simultaneously contacting the amino-acid residues at positions VAL312, and ILE328 of PPARδ (hPPARδ numbering). In some embodiments, a selective PPARδ agonist compound is capable of simultaneously contacting the amino-acid residues at positions VAL298, LEU303, VAL312, and ILE328 (hPPARδ numbering).

“Activation” herein is defined as the abovementioned downstream response, which in the case of PPAR's is gene transcription. Gene transcription, in some cases, is measured indirectly as downstream production of proteins reflective of the activation of the particular PPAR subtype under study. Alternatively, an artificial reporter construct, in some cases, is employed to study the activation of the individual PPAR's expressed in cells. The ligand binding domain of the particular receptor to be studied, in some cases, is fused to the DNA binding domain of a transcription factor, which produces convenient laboratory readouts, such as the yeast GAL4 transcription factor DNA binding domain. The fusion protein, in some cases, is transfected into a laboratory cell line along with a Gal4 enhancer, which effects the expression of the luciferase protein. When such a system is transfected into a laboratory cell line, binding of a receptor agonist to the fusion protein will result in light emission.

In some embodiments, a selective PPARδ agonist compound exemplifies the above gene transcription profile in cells selectively expressing PPARδ, and not in cells selectively expressing PPARγ or PPARα. In an embodiment, the cells express human PPARδ, PPARγ, and PPARα, respectively.

In a further embodiment, a PPARδ agonist compound may have an EC₅₀ value of less than about 5 μm as determined by the PPAR transient transactivation assay described below. In an embodiment, the EC₅₀ value is less than about 1 μm. In another embodiment, the EC₅₀ value is less than about 500 nM. In another embodiment, the EC₅₀ value is less than about 100 nM. In another embodiment, the EC₅₀ value is less than about 50 nM.

The PPAR transient transactivation assay, in some cases, is based on transient transfection into human HEK293 cells of two plasmids encoding a chimeric test protein and a reporter protein respectively. The chimeric test protein, in some cases, is a fusion of the DNA binding domain (DBD) from the yeast GAL4 transcription factor to the ligand binding domain (LBD) of the human PPAR proteins. The PPAR-LBD moiety harbors in addition to the ligand binding pocket also the native activation domain, allowing the fusion protein to function as a PPAR ligand dependent transcription factor. The GAL4 DBD directs the chimeric protein to bind only to Gal4 enhancers (of which none exist in HEK293 cells). The reporter plasmid contains a Gal4 enhancer driving the expression of the firefly luciferase protein. After transfection, HEK293 cells express the GAL4-DBD-PPAR-LBD fusion protein. The fusion protein will in turn bind to the Gal4 enhancer controlling the luciferase expression, and do nothing in the absence of ligand. Upon addition to the cells of a PPAR ligand, luciferase protein will be produced in amounts corresponding to the activation of the PPAR protein. The amount of luciferase protein is measured by light emission after addition of the appropriate substrate.

Cell Culture and Transfection: HEK293 cells, in some cases, are grown in DMEM+10% FCS. Cells, in some cases, are seeded in 96-well plates the day before transfection to give a confluency of 50-80% at transfection. A total of 0.8 mg DNA containing 0.64 mg pM1a/gLBD, 0.1 mg pCMVbGal, 0.08 mg pGL2(Gal4)₅,and 0.02 mg pADVANTAGE, in some cases, are transfected per well using FuGene transfection reagent according to the manufacturer's instructions. Cells, in some cases, are allowed to express protein for 48 hours followed by addition of compound.

Plasmids: Human PPARδ, in some cases, is obtained by PCR amplification using cDNA synthesized by reverse transcription of mRNA from human liver, adipose tissue, and plancenta, respectively. In some embodiments, amplified cDNAs are cloned into pCR2.1 and sequenced. The ligand binding domain (LBD) of each PPAR isoform, in some cases, is generated by PCR (PPARδ: aa 128—C-terminus) and fused to the DNA binding domain (DBD) of the yeast transcription factor GAL4 by subcloning fragments in frame into the vector pM1 (Sadowski et al. (1992), Gene 118, 137), generating the plasmids pM1αLBD, pM1γLBD, and pM16. Ensuing fusions, in some cases, are verified by sequencing. The reporter, in some cases, is constructed by inserting an oligonucleotide encoding five repeats of the GAL4 recognition sequence (Webster et al. (1988), Nucleic Acids Res. 16, 8192) into the vector pGL2 promotor (Promega), generating the plasmid pGL2(GAL4)₅. pCMVbGal, in some cases, is purchased from Clontech and pADVANTAGE, in some cases, is purchased from Promega.

Compounds: Compounds, in some cases, are dissolved in DMSO and diluted 1:1000 upon addition to the cells. Compounds, in some cases, are tested in quadruple in concentrations ranging from 0.001 to 300 μM. Cells, in some cases, are treated with compound for 24 h followed by luciferase assay. Each compound, in some cases, is tested in at least two separate experiments.

Luciferase assay: Medium including test compound, in some cases, is aspirated and 100 μl PBS including 1 mM Mg⁺⁺ and Ca⁺⁺, in some cases, is added to each well. In some embodiments, the luciferase assay is performed using the LucLite kit according to the manufacturer's instructions (Packard Instruments). Light emission, in some cases, is quantified by counting on a Packard LumiCounter. To measure β-galactosidase activity, 25 ml supernatant from each transfection lysate, in some cases, is transferred to a new microplate. In some embodiments, β-Galactosidase assays are performed in the microwell plates using a kit from Promega and read in a Labsystems Ascent Multiscan reader. The β-galactosidase data, in some cases, is used to normalize (transfection efficiency, cell growth, etc.) the luciferase data.

Statistical methods: The activity of a compound, in some cases, is calculated as fold induction compared to an untreated sample. In some embodiments, for each compound, the efficacy (maximal activity) is given as a relative activity compared to Wy14,643 for PPARα, rosiglitazone for PPARγ, and carbacyclin for PPARδ. The EC50 is the concentration giving 50% of maximal observed activity. EC50 values, in some cases, is calculated via non-linear regression using GraphPad PRISM 3.02 (GraphPad Software, San Diego, Calif.).

In a further embodiment, a PPARδ agonist compound has a molecular weight of less than about 1000 g/mol, or a molecular weight of less than about 950 g/mol, or a molecular weight of less than about 900 g/mol, or a molecular weight of less than about 850 g/mol, or a molecular weight of less than about 800 g/mol, or a molecular weight of less than about 750 g/mol, or a molecular weight of less than about 700 g/mol, or a molecular weight of less than about 650 g/mol, or a molecular weight of less than about 600 g/mol, or a molecular weight of less than about 550 g/mol, or a molecular weight of less than about 500 g/mol, or a molecular weight of less than about 450 g/mol, or a molecular weight of less than about 400 g/mol, or a molecular weight of less than about 350 g/mol, or a molecular weight of less than about 300 g/mol, or a molecular weight of less than about 250 g/mol. In another embodiment, a PPARδ agonist compound has a molecular weight of greater than about 200 g/mol, or a molecular weight of greater than about 250 g/mol, or a molecular weight of greater than about 250 g/mol, or a molecular weight of greater than about 300 g/mol, or a molecular weight of greater than about 350 g/mol, or a molecular weight of greater than about 400 g/mol, or a molecular weight of greater than about 450 g/mol, or a molecular weight of greater than about 500 g/mol, or a molecular weight of greater than about 550 g/mol, or a molecular weight of greater than about 600 g/mol, or a molecular weight of greater than about 650 g/mol, or a molecular weight of greater than about 700 g/mol, or a molecular weight of greater than about 750 g/mol, or a molecular weight of greater than about 800 g/mol, or a molecular weight of greater than about 850 g/mol, or a molecular weight of greater than about 900 g/mol, or a molecular weight of greater than about 950 g/mol, or a molecular weight of greater than about 1000 g/mol. In some embodiments, any of the upper and lower limits described above in this paragraph are combined.

In some embodiments, a PPARδ agonist compound is a PPARδ agonist compound disclosed in any of the following published patent applications: WO 97/027847, WO 97/027857, WO 97/028115, WO 97/028137, WO 97/028149, WO 98/027974, WO 99/004815, WO 2001/000603, WO 2001/025181, WO 2001/025226, WO 2001/034200, WO 2001/060807, WO 2001/079197, WO 2002/014291, WO 2002/028434, WO 2002/046154, WO 2002/050048, WO 2002/059098, WO 2002/062774, WO 2002/070011, WO 2002/076957, WO 2003/016291, WO 2003/024395, WO 2003/033493, WO 2003/035603, WO 2003/072100, WO 2003/074050, WO 2003/074051, WO 2003/074052, WO 2003/074495, WO 2003/084916, WO 2003/097607, WO 2004/000315, WO 2004/000762, WO 2004/005253, WO 2004/037776, WO 2004/060871, WO 2004/063165, WO 2004/063166, WO 2004/073606, WO 2004/080943, WO 2004/080947, WO 2004/092117, WO 2004/092130, WO 2004/093879, WO 2005/060958, WO 2005/097098, WO 2005/097762, WO 2005/097763, WO 2005/115383, WO 2006/055187, WO 2007/003581, and WO 2007/071766 (each of which is incorporated for such PPARδ agonist compounds).

In some embodiments, a PPARδ agonist compound is a PPARδ agonist compound disclosed in any of the following published patent applications: WO2014/165827; WO2016/057660; WO2016/057658; WO2017/180818; WO2017/062468; and WO/2018/067860 (each of which is incorporated for such PPARδ agonist compounds).

In some embodiments, a PPARδ agonist compound is a PPARδ agonist compound disclosed in any of the following published patent applications: United States Patent Application Publication Nos. 20160023991, 201 70226154, 20170304255, and 20170305894 (each of which is incorporated for such PPARδ agonist compounds).

In some embodiments, a PPARδ agonist compound is a phenoxyalkylcarboxylic acid compound. In some embodiments, the phenoxyalkylcarboxylic acid compound is a 2-methylphenoxyalkylcarboxylic acid compound.

In some embodiments, a PPARδ agonist compound is a phenoxyalkylcarboxylic acid compound that is a phenoxyethanoic acid compound, phenoxypropanoic acid compound, phenoxypropenoic acid compound, phenoxybutanoic acid compound, phenoxybutenoic acid compound, phenoxypentanoic acid compound, phenoxypentenoic acid compound, phenoxyhexanoic acid compound, phenoxyhexenoic acid compound, phenoxyoctanoic acid compound, phenoxyoctenoic acid compound, phenoxynonanoic acid compound, phenoxynonenoic acid compound, phenoxydecanoic acid compound, or phenoxydecenoic acid compound. In some embodiments, a PPARδ agonist compound is a phenoxyethanoic acid compound or a phenoxyhexanoic acid compound. In some embodiments, a PPARδ agonist compound is a phenoxyethanoic acid compound. In some embodiments, the phenoxyethanoic acid compound is a 2-methylphenoxyethanoic acid compound. In some embodiments, a PPARδ agonist compound is a phenoxyhexanoic acid compound.

In some embodiments, a PPARδ agonist compound is a phenoxyethanoic acid compound, a ((benzamidomethyl)phenoxy)hexanoic acid compound, a ((heteroarylmethyl)phenoxy)hexanoic acid compound, a methylthiophenoxyethanoic acid compound, or an allyloxyphenoxyethanoic acid compound.

In some embodiments, a PPARδ agonist compound is a ((benzamidomethyl)phenoxy)hexanoic acid compound.

In some embodiments, a PPARδ agonist compound is a ((heteroarylmethyl)phenoxy)hexanoic acid compound. In some embodiments, a PPARδ agonist compound is a ((imidazolylmethyl)phenoxy)hexanoic acid compound. In some embodiments, a PPARδ agonist compound is an imidazol-1-ylmethylphenoxyhexanoic acid compound. In some embodiments, a PPARδ agonist compound is a 6-(2-((2-phenyl-1H-imidazol-1-yl)methyl)phenoxy)hexanoic acid.

In some embodiments, a PPARδ agonist compound is an allyloxyphenoxyethanoic acid compound. In some embodiments, the allyloxyphenoxyethanoic acid compound is a 4-allyloxy-2-methylphenoxy)ethanoic acid compound.

In some embodiments, a PPARδ agonist compound is a methylthiophenoxyethanoic acid compound. In some embodiments, a PPARδ agonist compound is a 4-(methylthio)phenoxy)ethanoic acid compound.

In some embodiments, a PPARδ agonist compound is a phenoxyalkylcarboxylic acid compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid (Compound 1); (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; (R)-3-methyl-6-(2-((5-methyl-2-(4-(trifluoromethyl)phenyl)-1H-imidazol-1-yl)methyl)phenoxy)hexanoic acid; (R)-3-methyl-6-(2-((5-methyl-2-(6-(trifluoromethyl)pyridin-3-yl)-1H-imidazol-1-yl)methyl)phenoxy)hexanoic acid; (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025); (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); 2-(4-{2-[(4-Chlorobenzoyl)amino]ethyl}phenoxy)-2-methylpropanoic acid (bezafibrate); or a pharmaceutically acceptable salt thereof.

In another embodiment, a PPARδ agonist compound is a 2-methylphenoxyalkylcarboxylic acid compound selected from the group consisting of (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025).

In another embodiment, a PPARδ agonist compound is a compound selected from the group consisting of (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); and 2-(4-{2-[(4-Chlorobenzoyl)amino]ethyl}phenoxy)-2-methylpropanoic acid (bezafibrate).

In another embodiment, a PPARδ agonist compound is a compound selected from the group consisting of sodelglitazar; lobeglitazone; netoglitazone; and isaglitazone; 2-(4-{2-[(4-Chlorobenzoyl)amino]ethyl}phenoxy)-2-methylpropanoic acid (bezafibrate); 2-[2-methyl-4-[[3-methyl-4-[[4-(trifluoromethyl)phenyl]methoxy]phenyl]thio]phenoxy]-acetic acid (See WO 2003/024395); (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); {2-methyl-4-[5-methyl-2-(4-trifluoromethyl-phenyl)-2H-[1,2,3]triazol-4-ylmethylsylfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025).

In some embodiments, a PPARδ agonist compound is (E)-[4-[3-(4-Fluorophenyl)-3-[4-[3-(morpholin-4-yl)propynyl]phenyl]allyloxy]-2-methyl-phenoxy]acetic acid (Compound 1):

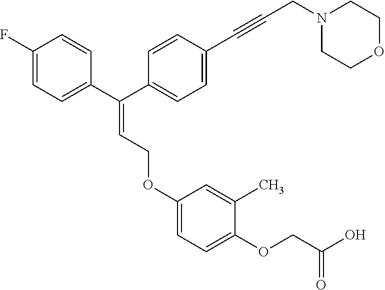 [SMILES: Cc1cc(OC/C=C(/c2ccc(F)cc2)c2ccc(C#CCN3CCOCC3)cc2)ccc1OCC(=O)O]

An example of the chemical synthesis of (E)-[4-[3-(4-Fluorophenyl)-3-[4-[3-(morpholin-4-yl)propynyl]phenyl]allyloxy]-2-methyl-phenoxy]acetic acid is found in Example 10 of PCT Application Pub. No. WO 2007/071766.

Compound 1 was tested on all three human PPAR subtypes (hPPAR): hPPARα, hPPARγ, and hPPARδ in vitro assays testing for such activity. Compound 1 exhibited a significantly greater selectivity for PPARδ over PPARα and PPARγ in humans, monkey, and mouse (see Table 1). In some cases, Compound 1 acts as a full agonist of PPARδ and only a partial agonist for both PPARα and PPARγ. In some cases, Compound 1 demonstrates negligible activity on PPARα and/or PPARγ in transactivation assays testing for such activity.

TABLE 1
Potency of Compound 1 in Muman, Monkey and
Mouse PPAR Receptor Transactivation Assays

| Species | PPAR Receptor Subtype | EC₅₀ (nM) Mean |
|---|---|---|
| Human | PPAR_{α} | >10,000 |
| Human | PPAR_{γ} | >10,000 |
| Human | PPAR_{δ} | 31 ± 3 |
| Monkey | PPAR_{α} | >1000 |
| Monkey | PPAR_{γ} | >1000 |
| Monkey | PPAR_{δ} | 6.6 |
| Mouse | PPAR_{α} | >10,000 |
| Mouse | PPAR_{γ} | >10,000 |
| Mouse | PPAR_{δ} | 240 |

In some embodiments, Compound 1 did not show any human retinoid X receptor (hRXR) activity, or activity on the nuclear receptors FXR, LXR_{α} or LXR_{β}. as a full agonist of PPARδ and only a partial agonist for both PPARα and PPARγ.

In some embodiments, a PPARδ agonist compound is (Z)-[2-Methyl-4-[3-(4-methylphenyl)-3-[4-[3-(morpholin-4-yl)propynyl]phenyl]allyloxy]-phenoxy]acetic acid:

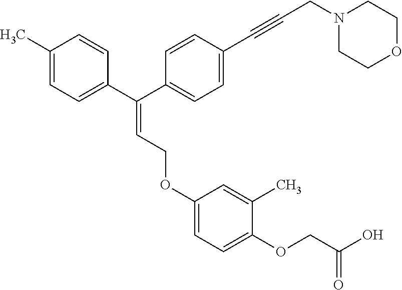 [SMILES: Cc1ccc(/C(=C/COc2ccc(OCC(=O)O)c(C)c2)c2ccc(C#CCN3CCOCC3)cc2)cc1]

An example of the chemical synthesis of (Z)-[2-Methyl-4-[3-(4-methylphenyl)-3-[4-[3-(morpholin-4-yl)propynyl]phenyl]allyloxy]-phenoxy]acetic acid is found in Example 3 of PCT Application Pub. No. WO 2007/071766.

In some embodiments, a PPARδ agonist compound is (E)-[2-Methyl-4-[3-[4-[3-(pyrazol-1-yl)prop-1-ynyl]phenyl]-3-(4-trifluoromethylphenyl)-allyloxy]phenoxy]acetic acid:

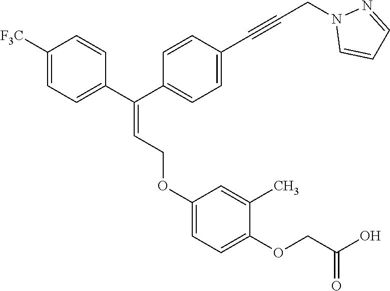 [SMILES: Cc1cc(OC/C=C(\c2ccc(C#CCn3cccn3)cc2)c2ccc(C(F)(F)F)cc2)ccc1OCC(=O)O]

An example of the chemical synthesis of (E)-[2-Methyl-4-[3-[4-[3-(pyrazol-1-yl)prop-1-ynyl]phenyl]-3-(4-trifluoromethylphenyl)-allyloxy]phenoxy]acetic acid is found in Example 4 of PCT Application Pub. No. WO 2007/071766.

In some embodiments, a PPARδ agonist compound is (E)-[2-Methyl-4-[3-[4-[3-(morpholin-4-yl)propynyl]phenyl]-3-(4-trifluoromethylphenyl)allyloxy]-phenoxy]acetic acid:

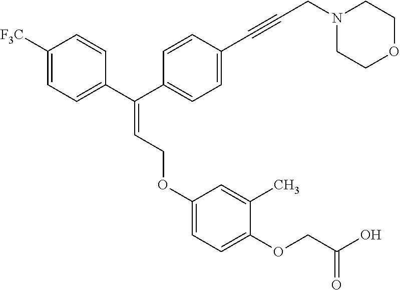 [SMILES: Cc1cc(OC/C=C(\c2ccc(C#CCN3CCOCC3)cc2)c2ccc(C(F)(F)F)cc2)ccc1OCC(=O)O]

An example of the chemical synthesis of (E)-[2-Methyl-4-[3-[4-[3-(morpholin-4-yl)propynyl]phenyl]-3-(4-trifluoromethylphenyl)allyloxy]-phenoxy]acetic acid is found in Example 20 of PCT Application Pub. No. WO 2007/071766.

In some embodiments, a PPARδ agonist compound is (E)-[4-[3-(4-Chlorophenyl)-3-[4-[3-(morpholin-4-yl)propynyl]phenyl]allyloxy]-2-methyl-phenoxy]acetic acid:

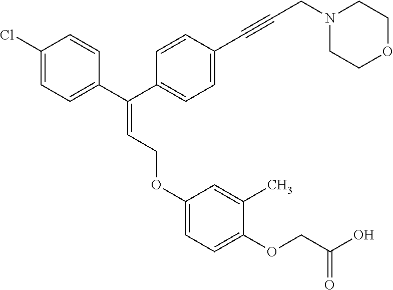 [SMILES: Cc1cc(OC/C=C(/c2ccc(Cl)cc2)c2ccc(C#CCN3CCOCC3)cc2)ccc1OCC(=O)O]

An example of the chemical synthesis of (E)-[4-[3-(4-Chlorophenyl)-3-[4-[3-(morpholin-4-yl)propynyl]phenyl]allyloxy]-2-methyl-phenoxy]acetic acid is found in Example 46 of PCT Application Pub. No. WO 2007/071766.

In some embodiments, a PPARδ agonist compound is (E)-[4-[3-(4-Chlorophenyl)-3-[4-[3-(morpholin-4-yl)propynyl]phenyl]allyloxy]-2-methylphenyl]-propionic acid:

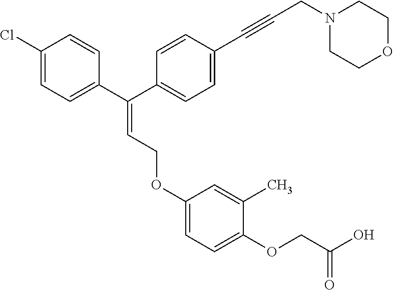 [SMILES: Cc1cc(OC/C=C(/c2ccc(Cl)cc2)c2ccc(C#CCN3CCOCC3)cc2)ccc1OCC(=O)O]

An example of the chemical synthesis of (E)-[4-[3-(4-Chlorophenyl)-3-[4-[3-(morpholin-4-yl)propynyl]phenyl]allyloxy]-2-methylphenyl]-propionic acid is found in Example 63 of PCT Application Pub. No. WO 2007/071766.

In some embodiments, a PPARδ agonist compound is {4-[3,3-Bis-(4-bromo-phenyl)-allyloxy]-2-methyl-phenoxy}-acetic acid:

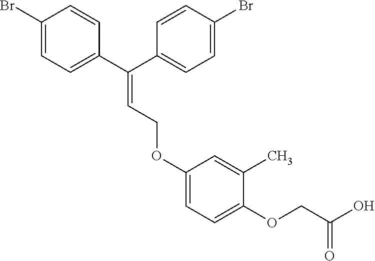 [SMILES: Cc1cc(OCC=C(c2ccc(Br)cc2)c2ccc(Br)cc2)ccc1OCC(=O)O]

An example of the chemical synthesis of {4-[3,3-Bis-(4-bromo-phenyl)-allyloxy]-2-methyl-phenoxy}-acetic acid is found in Example 10 of PCT Application Pub. No. WO 2004/037776.

In some embodiments, a PPARδ agonist compound is {4-[3-Isobutoxy-5-(3-morpholin-4-yl-prop-1-ynyl)-benzylsulfanyl]-2-methyl-phenoxy}-acetic acid:

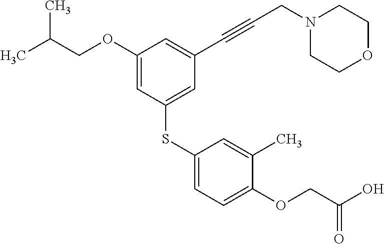 [SMILES: Cc1cc(Sc2cc(C#CCN3CCOCC3)cc(OCC(C)C)c2)ccc1OCC(=O)O]

An example of the chemical synthesis of {4-[3-Isobutoxy-5-(3-morpholin-4-yl-prop-1-ynyl)-benzylsulfanyl]-2-methyl-phenoxy}-acetic acid is found in Example 9 of PCT Application Pub. No. WO 2007/003581.

In some embodiments, a PPARδ agonist compound is {4-[3-Isobutoxy-5-(3-morpholin-4-yl-prop-1-ynyl)-phenylsulfanyl]-2-methyl-phenoxy}-acetic acid:

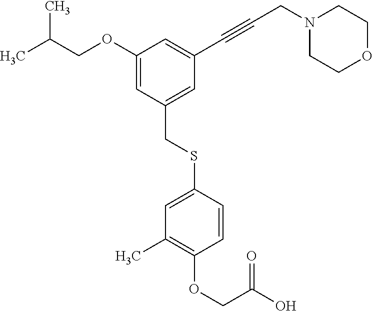 [SMILES: Cc1cc(SCc2cc(C#CCN3CCOCC3)cc(OCC(C)C)c2)ccc1OCC(=O)O]

An example of the chemical synthesis of {4-[3-Isobutoxy-5-(3-morpholin-4-yl-prop-1-ynyl)-phenylsulfanyl]-2-methyl-phenoxy}-acetic acid is found in Example 35 of PCT Application Pub. No. WO 2007/003581.

Accordingly, in an embodiment, a PPARδ agonist compound is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; or a pharmaceutically acceptable salt thereof.

In a further embodiment, a PPARδ agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof. In some embodiments, the PPARδ agonist compound is (E)-[4-[3-(4-Fluorophenyl)-3-[4-[3-(morpholin-4-yl)propynyl]phenyl]allyloxy]-2-methyl-phenoxy]acetic acid sodium salt.

In a further embodiment, a PPARδ agonist compound is Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16, disclosed in Wu et al. Proc Natl Acad Sci USA Mar. 28, 2017 114 (13) E2563-E2570.

In a further embodiment, a PPARδ agonist compound is (R)-3-methyl-6-(2-((5-methyl-2-(4-(trifluoromethyl)phenyl)-1H-imidazol-1-yl)methyl)phenoxy)hexanoic acid, or (R)-3-methyl-6-(2-((5-methyl-2-(6-(trifluoromethyl)pyridin-3-yl)-1H-imidazol-1-yl)methyl)phenoxy)hexanoic acid, or a pharmaceutically acceptable salt thereof.

In a further embodiment, a PPARδ agonist compound is (R)-3-methyl-6-(2-((5-methyl-2-(4-(trifluoromethyl)phenyl)-1H-imidazol-1-yl)methyl)phenoxy)hexanoic acid, or a pharmaceutically acceptable salt thereof. In some embodiments, the PPARδ agonist compound is the hemisulfate salt of (R)-3-methyl-6-(2-((5-methyl-2-(4-(trifluoromethyl)phenyl)-1H-imidazol-1-yl)methyl)phenoxy)hexanoic acid. In some embodiments, the PPARδ agonist compound is the meglumine salt of (R)-3-methyl-6-(2-((5-methyl-2-(4-(trifluoromethyl)phenyl)-1H-imidazol-1-yl)methyl)phenoxy)hexanoic acid.

In a further embodiment, a PPARδ agonist compound is (R)-3-methyl-6-(2-((5-methyl-2-(6-(trifluoromethyl)pyridin-3-yl)-1H-imidazol-1-yl)methyl)phenoxy)hexanoic acid, or a pharmaceutically acceptable salt thereof. In some embodiments, the PPARδ agonist compound is the hemisulfate salt of (R)-3-methyl-6-(2-((5-methyl-2-(6-(trifluoromethyl)pyridin-3-yl)-1H-imidazol-1-yl)methyl)phenoxy)hexanoic acid. In some embodiments, the PPARδ agonist compound is the meglumine salt of (R)-3-methyl-6-(2-((5-methyl-2-(6-(trifluoromethyl)pyridin-3-yl)-1H-imidazol-1-yl)methyl)phenoxy)hexanoic acid.

In a further embodiment, a PPARδ agonist compound is 2-(2-methyl-4-(((2-(4-(trifluoromethyl)phenyl)-2H-1,2,3-triazol-4-yl)methyl)thio)phenoxy)acetic acid, or a pharmaceutically acceptable salt thereof.

In a further embodiment, a PPARδ agonist compound is (R)-2-(4-((2-ethoxy-3-(4-(trifluoromethyl)phenoxy)propyl)thio)phenoxy)acetic acid, or a pharmaceutically acceptable salt thereof.

The term “pharmaceutically acceptable salt” in reference to a PPARδ agonist compound refers to a salt of the PPARδ agonist compound, which does not cause significant irritation to a mammal to which it is administered and does not substantially abrogate the biological activity and properties of the compound. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. In some embodiments, pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability, in some cases, is manipulated as one aspect of delayed and sustained release behaviors. Also, because the salt-forming molecule, in some cases, is in equilibrium with a neutral form, passage through biological membranes, in some cases, is adjusted.

In some embodiments, pharmaceutically acceptable salts are generally prepared by reacting the free base with a suitable organic or inorganic acid or by reacting the acid with a suitable organic or inorganic base. The term may be used in reference to any compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. In some embodiments, when an acidic substituent is present, such as —CO₂H, ammonium, morpholinium, sodium, potassium, barium, or calcium salts, and the like are formed. In some embodiments, when a basic group is present, such as amino, or a basic heteroaryl ring, such as pyridyl, an acidic addition salt is formed, such as hydrochloride salt, hydrobromide salt, phosphate salt, sulfate salt, trifluoroacetate salt, trichloroacetate salt, acetate salt, oxalate salt, maleate salt, pyruvate salt, malonate salt, succinate salt, citrate salt, tartarate salt, fumarate salt, mandelate salt, benzoate salt, cinnamate salt, methanesulfonate salt, ethanesulfonate salt, picrate salt, and the like. Additional pharmaceutically acceptable salt forms of therapeutic agents are listed in Berge, et al., Journal of PharmaceuticalSciences, Vol. 66(1), pp. 1-19 (1977).

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof.

In some embodiments, a modulator is an antagonist. In some embodiments, a modulator is a degrader.

The terms “administer,” “administering”, “administration,” and the like, as used herein, refer to the methods that in some cases enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration.

Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein is carried out by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route, in some cases, depends upon for example the condition and disorder of the recipient. By way of example only, compounds described herein, in some cases, are administered locally to the area in need of treatment, by for example, local infusion during surgery, topical application such as creams or ointments, injection, catheter, or implant. In some cases, administration occurs by direct injection at the site of a diseased tissue or organ.

In some embodiments, a PPARδ agonist compound is included within a pharmaceutical composition. As used herein, the term “pharmaceutical composition” refers to a liquid or solid composition that contains a pharmaceutically active ingredient (e.g., a PPARδ agonist compound) and at least a carrier, where none of the ingredients is generally biologically undesirable at the administered quantities.

Pharmaceutical compositions incorporating a PPARδ agonist compound take any physical form that is pharmaceutically acceptable. In some embodiments, pharmaceutical compositions described herein are in a suitable form for oral administration. In one embodiment of such pharmaceutical compositions, a therapeutically effective amount of a PPARδ agonist compound is incorporated.

In some embodiments, conventional inert ingredients and manner of formulating the pharmaceutical compositions are used. In some embodiments, known methods of formulating the pharmaceutical compositions are followed. All of the usual types of compositions are contemplated, including, but not limited to, tablets, chewable tablets, capsules, and solutions. The amount of the PPARδ agonist compound, however, is best defined as the effective amount, that is, the amount of the PPARδ agonist compound that provides the desired dose to the subject in need of such treatment. In some embodiments, the activity of the PPARδ agonist compound does not depend on the nature of the composition, so the compositions are chosen and formulated solely for convenience and economy. Any of the PPARδ agonist compounds as described herein are formulated in any desired form of composition.

In some cases, capsules are prepared by mixing the PPARδ agonist compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

In some cases, tablets are prepared by direct compression, by wet granulation, or by dry granulation. Their formulations usually incorporate diluents, binders, lubricants, and disintegrators, as well as the PPARδ agonist compound. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin, and sugars such as lactose, fructose, glucose, and the like. Natural and synthetic gums are also convenient, including acacia, alginates, methylcellulose, polyvinylpyrrolidine, and the like. In some cases, polyethylene glycol, ethylcellulose, and waxes serve as binders.

In some cases, a lubricant in a tablet formulation helps prevent the tablet and punches from sticking in the die. In some cases, a lubricant is chosen from such solids as talc, magnesium and calcium stearate, stearic acid, and hydrogenated vegetable oils.

Tablet disintegrators are substances that swell when wetted to break up the tablet and release the compound. They include starches, clays, celluloses, aligns, and gums. More particularly, tablet disintegrators include corn and potato starches, methylcellulose, agar, bentonite, wood cellulose, powdered natural sponge, cation-exchange resins, alginic acid, guar gum, citrus pulp, carboxymethylcellulose, and sodium lauryl sulfate.

Enteric formulations are often used to protect an active ingredient from the strongly acidic contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. In some cases, the PPARδ agonist compound is formulated as chewable tablets by using large amounts of pleasant-tasting substances such as mannitol in the formulation.

In some cases, transdermal patches are used to deliver the PPARδ agonist compound. Typically, a patch comprises a resinous composition in which the active compound(s) will dissolve, or partially dissolve, and is held in contact with the skin by a film that protects the composition. Other, more complicated patch compositions are also in use, particularly those having a membrane pierced with innumerable pores through which the drugs are pumped by osmotic action.

In any embodiment where a PPARδ agonist compound is included in a pharmaceutical composition, such pharmaceutical compositions, in some cases, are in a form suitable for oral use, for example, as tablets, troches, lozenges, aqueous or oily suspensions, dispersible powders or granules, emulsions, hard or soft capsules, or syrups or elixirs.

Compositions intended for oral use, in some cases, are prepared according to any known method, and such compositions, in some cases, contain one or more agents selected from the group consisting of sweetening agents, flavoring agents, coloring agents, and preserving agents in order to provide pharmaceutically elegant and palatable preparations. Tablets, in some cases, contain the active ingredient in admixture with non-toxic pharmaceutically acceptable excipients that are suitable for the manufacture of tablets. These excipients include for example, inert diluents, such as calcium carbonate, sodium carbonate, lactose, calcium phosphate, or sodium phosphate; granulating and disintegrating agents, for example, corn starch or alginic acid; binding agents, for example, starch, gelatin, or acacia; and lubricating agents, for example, magnesium stearate, stearic acid, or talc. The tablets, in some cases, are uncoated or they, in some cases, are coated by known techniques to delay disintegration and absorption in the gastrointestinal tract and thereby provide a sustained action over a longer period. For example, a time delay material such as glyceryl monostearate or glyceryl distearate, in some cases, is employed.

### Methods of Dosing and Treatment Regimens

In one embodiment, a PPARδ agonist compound (e.g. Compound 1, or a pharmaceutically acceptable salt thereof), is used in the preparation of medicaments for the treatment of primary mitochondrial myopathy in a mammal. Methods for treating any of the diseases or conditions described herein in a mammal in need of such treatment, involves administration of pharmaceutical compositions that include a PPARδ agonist compound (e.g. Compound 1, or a pharmaceutically acceptable salt thereof), active metabolite, prodrug, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing a PPARδ agonist compound (e.g. Compound 1, or a pharmaceutically acceptable salt thereof), are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a PPARδ agonist compound (e.g. Compound 1, or a pharmaceutically acceptable salt thereof), in order to prevent a return of the symptoms of the disease or condition.

In certain embodiments wherein the patient's condition does not improve, upon the doctor's discretion the administration of a PPARδ agonist compound (e.g. Compound 1, or a pharmaceutically acceptable salt thereof), is administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between about 2 days and about 1 year, including by way of example only, about 2 days, about 3 days, about 4 days, about 5 days, about 6 days, about 7 days, about 10 days, about 12 days, about 15 days, about 20 days, about 28 days, or more than about 28 days. The dose reduction during a drug holiday is, by way of example only, by about 10%-100%, including by way of example only about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

In one aspect, a PPARδ agonist compound (e.g. Compound 1, or a pharmaceutically acceptable salt thereof), is administered daily to humans in need of therapy a PPARδ agonist compound (e.g. Compound 1, or a pharmaceutically acceptable salt thereof). In some embodiments, a PPARδ agonist compound (e.g. Compound 1, or a pharmaceutically acceptable salt thereof), is administered once a day. In some embodiments, a PPARδ agonist compound (e.g. Compound 1, or a pharmaceutically acceptable salt thereof), is administered twice a day. In some embodiments, a PPARδ agonist compound (e.g. Compound 1, or a pharmaceutically acceptable salt thereof), is administered three times a day. In some embodiments, a PPARδ agonist compound (e.g. Compound 1, or a pharmaceutically acceptable salt thereof), is administered every other day. In some embodiments, a PPARδ agonist compound (e.g. Compound 1, or a pharmaceutically acceptable salt thereof), is administered twice a week.

In some instances, a PPARδ agonist compound (e.g. Compound 1, or a pharmaceutically acceptable salt thereof) is administered once per day, twice per day, three times per day or more. In some instances, a PPARδ agonist compound (e.g. Compound 1, or a pharmaceutically acceptable salt thereof) is administered twice per day. A PPARδ agonist compound (e.g. Compound 1, or a pharmaceutically acceptable salt thereof), in some embodiments, is administered daily, every day, every alternate day, five days a week, once a week, every other week, two weeks per month, three weeks per month, once a month, twice a month, three times per month, or more. In some embodiments, a PPARδ agonist compound (e.g. Compound 1, or a pharmaceutically acceptable salt thereof) is administered twice daily, e.g., morning and evening. In some embodiments, a PPARδ agonist compound (e.g. Compound 1, or a pharmaceutically acceptable salt thereof) is administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, a PPAR-delta agonist (e.g. Compound 1, or a pharmaceutically acceptable salt thereof) is administered twice daily for at least or about 1 week, 2 weeks, 3 weeks, 1 month, 2 months, 3 months, 4 months, 5 months, 6 months, or more. In some embodiments, a PPARδ agonist compound (e.g. Compound 1, or a pharmaceutically acceptable salt thereof) is administered once daily, twice daily, three times daily, four times daily, or more than four times daily for at least or about 1 week, 2 weeks, 3 weeks, 1 month, 2 months, 3 months, 4 months, 5 months, 6 months, or more.

In general, doses of a PPARδ agonist compound (e.g. Compound 1, or a pharmaceutically acceptable salt thereof), employed for treatment of the diseases or conditions described herein in humans are typically in the range of from about 0.1 mg to about 10 mg/kg of body weight per dose. In one embodiment, the desired dose is conveniently presented in a single dose or in divided doses administered simultaneously (or over a short period of time) or at appropriate intervals, for example as two, three, four or more sub-doses per day. In some embodiments, a PPARδ agonist compound (e.g. Compound 1, or a pharmaceutically acceptable salt thereof), is conveniently presented in divided doses that are administered simultaneously (or over a short period of time) once a day. In some embodiments, a PPARδ agonist compound (e.g. Compound 1, or a pharmaceutically acceptable salt thereof), is conveniently presented in divided doses that are administered in equal portions twice-a-day.

In some embodiments, a PPARδ agonist compound (e.g. Compound 1, or a pharmaceutically acceptable salt thereof), is administered orally to the human at a dose from about 0.1 mg to about 10 mg/kg of body weight per dose. In some embodiments, a PPARδ agonist compound (e.g. Compound 1, or a pharmaceutically acceptable salt thereof), is administered to the human on a continuous dosing schedule. In some embodiments, a PPARδ agonist compound (e.g. Compound 1, or a pharmaceutically acceptable salt thereof), is administered to the human on a continuous daily dosing schedule.

The term “continuous dosing schedule” refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day. In some embodiments, daily administration is three times a day. In some embodiments, daily administration is more than three times a day.

The term “continuous daily dosing schedule” refers to the administration of a particular therapeutic agent everyday at roughly the same time each day. In some embodiments, daily administration is once a day. In some embodiments, daily administration is twice a day. In some embodiments, daily administration is three times a day. In some embodiments, daily administration is more than three times a day.

In some embodiments, the amount of a PPARδ agonist compound (e.g. Compound 1, or a pharmaceutically acceptable salt thereof), is administered once-a-day. In some other embodiments, the amount of a PPARδ agonist compound (e.g. Compound 1, or a pharmaceutically acceptable salt thereof), is administered twice-a-day. In some other embodiments, the amount of a PPARδ agonist compound (e.g. Compound 1, or a pharmaceutically acceptable salt thereof), is administered three times a day.

In certain embodiments wherein improvement in the status of the disease or condition in the human is not observed, the daily dose of a PPARδ agonist compound (e.g. Compound 1, or a pharmaceutically acceptable salt thereof), is increased. In some embodiments, a once-a-day dosing schedule is changed to a twice-a-day dosing schedule. In some embodiments, a three times a day dosing schedule is employed to increase the amount of a PPARδ agonist compound (e.g. Compound 1, or a pharmaceutically acceptable salt thereof), that is administered. In some embodiments, the frequency of administration by inhalation is increased in order to provide repeat high Cmax levels on a more regular basis. In some embodiments, the frequency of administration is increased in order to provide maintained or more regular exposure to a PPARδ agonist compound (e.g. Compound 1, or a pharmaceutically acceptable salt thereof). In some embodiments, the frequency of administration is increased in order to provide repeat high Cmax levels on a more regular basis and provide maintained or more regular exposure to a PPARδ agonist compound (e.g. Compound 1, or a pharmaceutically acceptable salt thereof).

In any of the aforementioned aspects are further embodiments comprising single administrations of the effective amount of a PPARδ agonist compound (e.g. Compound 1, or a pharmaceutically acceptable salt thereof), including further embodiments in which the PPARδ agonist compound, is administered (i) once a day; or (ii) multiple times over the span of one day.

In any of the aforementioned aspects are further embodiments comprising multiple administrations of the effective amount of a PPARδ agonist compound (e.g. Compound 1, or a pharmaceutically acceptable salt thereof), including further embodiments in which (i) the PPARδ agonist compound is administered continuously or intermittently: as in a single dose; (ii) the time between multiple administrations is every 6 hours; (iii) the PPARδ agonist compound is administered to the mammal every 8 hours; (iv) the PPARδ agonist compound is administered to the mammal every 12 hours; (v) the PPARδ agonist compound is administered to the mammal every 24 hours. In further or alternative embodiments, the method comprises a drug holiday, wherein the administration of the PPARδ agonist compound is temporarily suspended or the dose of the PPARδ agonist compound being administered is temporarily reduced; at the end of the drug holiday, dosing of the PPARδ agonist compound is resumed. In one embodiment, the length of the drug holiday varies from 2 days to 1 year.

Generally, a suitable dose of a PPARδ agonist compound, or a pharmaceutically acceptable salt thereof, for administration to a human will be in the range of about 0.1 mg/kg per day to about 25 mg/kg per day (e.g., about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 2 mg/kg per day, about 3 mg/kg per day, about 4 mg/kg per day, about 5 mg/kg per day, about 6 mg/kg per day, about 7 mg/kg per day, about 8 mg/kg per day, about 9 mg/kg per day, about 10 mg/kg per day, about 15 mg/kg per day, about 20 mg/kg per day, or about 25 mg/kg per day). Alternatively, a suitable dose of a PPARδ agonist compound, or a pharmaceutically acceptable salt thereof, for administration to a human will be in the range of from about 0.1 mg/day to about 1000 mg/day; from about 1 mg/day to about 400 mg/day; or from about 1 mg/day to about 300 mg/day. In other embodiments, a suitable dose of a PPARδ agonist compound, or a pharmaceutically acceptable salt thereof, for administration to a human will be about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. In some embodiments, dosages are administered more than one time per day (e.g., two, three, four, or more times per day). In one embodiment, a suitable dose of a PPARδ agonist compound, or a pharmaceutically acceptable salt thereof, for administration to a human is about 100 mg twice/day (i.e., a total of about 200 mg/day). In another embodiment, a suitable dose of a PPARδ agonist compound, or a pharmaceutically acceptable salt thereof, for administration to a human is about 50 mg twice/day (i.e., a total of about 100 mg/day).

In some embodiments, a suitable dose of Compound 1, or a pharmaceutically acceptable salt thereof, for administration to a human with a primary mitochondrial myopathy will be about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. In some embodiments, a suitable dose Compound 1, or a pharmaceutically acceptable salt thereof, for administration to a human will be about 50 mg/day, about 100 mg/day, about 150 mg/day, about 200 mg/day, about 250 mg/day, about 300 mg/day, about 350 mg/day, about 400 mg/day, about 450 mg/day, or about 500 mg/day. In some embodiments, a suitable dose of Compound 1, or a pharmaceutically acceptable salt thereof, for administration to a human will be about 50 mg/day. In some embodiments, a suitable dose of Compound 1, or a pharmaceutically acceptable salt thereof, for administration to a human will be about 100 mg/day. In some embodiments, dosages are administered more than one time per day (e.g., two, three, four, or more times per day).

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the individual subject, the severity of the disease or condition being treated, the identity (e.g., weight) of the human, and the particular additional therapeutic agents that are administered (if applicable), and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the PPARδ agonist compound lies within a range of circulating concentrations that include the ED₅₀ with minimal toxicity. In certain embodiments, the daily dosage range and/or the unit dosage amount varies within this range depending upon the dosage form employed and the route of administration utilized.

In some embodiments, following the administration of a therapeutically effective dose of the PPARδ agonist compound to a subject, the no observed adverse effect level (NOAEL) is at least 1, 10, 20, 50, 100, 500 or 1000 milligrams of the PPARδ agonist compound per kilogram of body weight (mpk). In some examples, the 7-day NOAEL for a rat administered PPARδ agonist compound is at least about 200, 300, 400, 500, 600, 700, 800, 900, 1000, 1500 or 2000 mpk. In some examples, the 7-day NOAEL for a dog administered PPARδ agonist compound is at least about 10, 20, 30, 40, 50, 60, 70, 80, 90, 100, 200, 500 mpk.

In some embodiments, a diagnosis of primary mitochondrial myopathy in a mammal is confirmed with a tissue biopsy and molecular genetic testing (e.g. Parikh S, et al. Diagnosis and management of mitochondrial disease: a consensus statement from the Mitochondrial Medicine Society. Genet Med. 2015; 17(9):689-701. doi:10.1038/gim.2014.177).

A human mitochondrial genome database is known, see for example, MITOMAP, a compendium of polymorphisms and mutations in human mitochondrial DNA. See also, Revised Cambridge Reference Sequence (rCRS) of the Human Mitochondrial DNA.

A tissue biopsy involves taking a small sample of affected tissue that is studied under a microscope. In some embodiments, chemical tests conducted on the tissue sample are also performed.

In some embodiments, a tissue biopsy comprises a muscle biopsy. In some embodiments, a variety of histological, biochemical, and genetic studies are performed on the tissue. Tissue testing allows for, but not limited to, detection of mtDNA mutations with tissue specificity or low-level heteroplasmy and quantification of mtDNA content (copy number).

In some embodiments, muscle histology includes, but is not limited to, hematoxylin and eosin (H&E), Gomori trichrome (for ragged red fibers), SDH (for SDH-rich or ragged blue fibers), NADH-TR (NADH-tetrazolium reductase), COX (for COX negative fibers), and combined SDH/COX staining (COX intermediate fibers). Electron microscopy (EM) examines the mitochondria for inclusions and ultrastructural abnormalities.

In some embodiments, functional in vitro assays in tissue (typically muscle) are performed to measure mitochondrial function. These tests evaluate the various functions of the mitochondrial ETC or respiratory chain. Functional assays include enzyme activities of the individual components of the ETC, measurements of the activity of components, blue-native gel electrophoresis, measurement of the presence of various protein components within complexes and supercomplexes (achieved via western blots and gel electrophoresis), and consumption of oxygen using various substrates and inhibitors.

In some embodiments, methods for treating primary mitochondrial myopathy in a mammal with a PPARδ agonist compound described herein (e.g. Compound 1, or a pharmaceutically acceptable salt thereof) results in improvements in muscle histology, increases in mitochondrial DNA copy number, improvements in heteroplasmy level, improvement (e.g. increases) in respiratory chain enzyme activity (such as, but not limited to, ATP-ADP levels, fatty acid oxidation gene expression or flux), and increases in mRNA levels (e.g. measured using transcriptomics).

In some embodiments, methods for treating a primary mitochondrial myopathy in a mammal with a PPARδ agonist compound described herein (e.g. Compound 1, or a pharmaceutically acceptable salt thereof) results in histological improvements in biopsied muscle samples taken from a mammal with a primary mitochondrial myopathy. In some embodiments, histological improvements in biopsied muscle samples comprises increasing the quality of mitochondria. In some embodiments, histological improvements in biopsied muscle samples comprises decreases in ragged red fibers.

In some embodiments, histological improvements in biopsied muscle samples improve by at least or about 10%, 15%, 20%, 25%, 30%, 35%, 40%, 45%, 50%, 55%, 60%, 65%, 70%, 75%, 80%, 85%, 90%, 95%, or more than 95%.

In some embodiments, mitochondrial DNA copy number increase by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the administration of a PPARδ agonist compound described herein (e.g. Compound 1, or a pharmaceutically acceptable salt thereof) to a mammal with primary mitochondrial myopathy results in mitochondrial DNA copy number improving by at least or about 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold.

In some embodiments, heteroplasmy levels improve by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the administration of a PPARδ agonist compound described herein (e.g. Compound 1, or a pharmaceutically acceptable salt thereof) to a mammal with primary mitochondrial myopathy results in heteroplasmy levels improving by at least or about 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold.

In some embodiments, respiratory chain enzyme activity improves by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the administration of a PPARδ agonist compound described herein (e.g. Compound 1, or a pharmaceutically acceptable salt thereof) to a mammal with primary mitochondrial myopathy results in respiratory chain enzyme improving by at least or about 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold.

Improvements, in some embodiments, are compared to a control. In some embodiments, a control is an individual who does not receive a PPARδ agonist (e.g. Compound 1, or a pharmaceutically acceptable salt thereof). In some embodiments, the control is an individual who does not receive a full dose of a PPARδ agonist (e.g. Compound 1, or a pharmaceutically acceptable salt thereof). In some embodiments, the control is baseline for the individual prior to receiving a PPARδ agonist (e.g. Compound 1, or a pharmaceutically acceptable salt thereof).

In some embodiments, methods for treating a primary mitochondrial myopathy in a mammal with a peroxisome proliferator-activated receptor delta (PPARδ) agonist compound described herein (e.g. Compound 1, or a pharmaceutically acceptable salt thereof) results in improvements in one or more outcome measures. In some embodiments, outcomes measures include, but are not limited to: patient reported outcomes (PRO), exercise tolerance, whole body fatty acid oxidation (e.g. ¹³CO₂ production), blood acylcarnitines profiles, and blood inflammatory cytokines. In some embodiments, a baseline assessment is determined, typically prior to the administration of a PPARδ agonist compound (e.g. Compound 1, or a pharmaceutically acceptable salt thereof). Improvements in outcome measures are assessed with repeated assessments taken during treatment with a PPARδ agonist compound and a comparison against the baseline assessment and/or any prior assessment(s).

In some embodiments, improvements in one or more outcome measures are by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the administration of a PPARδ agonist compound described herein (e.g. Compound 1, or a pharmaceutically acceptable salt thereof) to a mammal with primary mitochondrial myopathy results in one or more outcome measures improving by at least or about 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold.

In some embodiments, patient reported outcomes (PRO) are measured with questionnaires. In some embodiments, the questionnaire covers health concepts related to the disorder being treated. In some embodiments, the questionnaire covers health concepts related to the disorder being treated such as, but not, limited to: physical functioning, bodily pain, role limitations due to physical health problems, role limitations due to personal or emotional problems, emotional well-being, social functioning, energy/fatigue, and general health perceptions, including perceptions in change of health.

In some embodiments, outcome measures are assessed with tests that assess exercise tolerance or endurance. In some embodiments, exercise tolerance is assessed with exercise tests. Exercise tests include, but are not limited to, submaximal treadmill, walking tests (e.g. 6 minute; 12 minute), run tests, treadmill and ergometry exercise testing. In some embodiments, exercise tests are used in combination with the Borg Scale of perceived exertion. In some embodiments, exercise tests are performed according to guidelines set forth by the American Thoracic Society (ATS).

In some embodiments, treating primary mitochondrial myopathy comprises improving the mammal's exercise tolerance, decreasing pain, decreasing fatigue, increasing strength, increasing survival or a combination thereof.

In humans treating primary mitochondrial myopathy comprises improving a person's sense of feeling well, cognition, exercise tolerance, decreasing pain, decreasing fatigue, increasing strength, increasing survival, or a combination thereof.

In some embodiments, an improvement in a person's sense of well-being, pain, fatigue, and/or cognition is determined by asking the person treated to compare the aforementioned symptoms after treatment as compared to before treatment.

In some embodiments, an improvement in a person's symptoms can be determined by asking a caregiver to compare the subject's symptoms before and after treatment.

In some embodiments, improving the mammal's exercise tolerance comprises increasing endurance/exercise tolerance as measured by sit-stand tests, or the distance walked in a walking test such as about a 6-minute walk test or in a 12-minute walk test. In some embodiments, the distance walked in such a walking test increases by at least about 1 meter, at least about 5 meters, at least about 10 meters, at least about 20 meters, at least about 30 meters, at least about 40 meters, at least about 50 meter, at least about 60 meters, at least about 70 meters, at least about 80 meters, at least about 90 meters, at least about 100 meters, or more than about 100 meters.

As used herein the term “about” means within +10% of the value.

In some embodiments, improving the mammal's exercise tolerance comprises decreases in heart rate during the 12-minute walk test. In some embodiments, heart rate decreases by 1 heart beat per minute, by 2 heart beats per minute, by 3 heart beats per minute, by 4 heart beats per minute, by 5 heart beats per minute, by at least about 10 heart beats per minute, or by at least about 20 heart beats per minute.

In some embodiments, improving the mammal's exercise tolerance comprises increasing the mammal's peak or maximal uptake of oxygen (peak VO₂ or VO₂ max). VO₂ max, also known as maximal oxygen uptake, is the measurement of the maximum amount of oxygen a person can utilize during intense exercise. It is a common measurement used to establish the aerobic capacity of a person prior to or during the course of exercise.

In some embodiments, peak VO₂ is expressed either as an absolute rate (for example, litres of oxygen per minute (e.g. L/min)) or as a relative rate (for example, millilitres of oxygen per kilogram of body mass per minute (e.g., mL/min/kg min)).

In some embodiments, improving the mammal's exercise tolerance comprises increasing the mammals peak VO₂ measurement by about 0.5 mL/min/kg, by about 1 mL/min/kg, by about 1.5 mL/min/kg, by about 2 mL/min/kg, by about 2.5 mL/min/kg, by about 3 mL/min/kg, by about 3.5 mL/min/kg, by about 4 mL/min/kg, by about 4.5 mL/min/kg, by about 5 mL/min/kg, or more than about 5 mL/min/kg.

In some embodiments, improving the mammal's exercise tolerance comprises decreases in measured respiratory exchange ratios (RER).

In some embodiments, the respiratory exchange ratio (RER) is measured to assess exercise tolerance. RER is the ratio between the amount of carbon dioxide (CO₂) produced in metabolism and oxygen (O₂) used. In some embodiments, the ratio is determined by comparing exhaled gases to room air.

In some embodiments, a mammal's pain is evaluated with a Brief Pain Inventory (BPI). BPI comprises a questionnaire that assesses the severity of pain and the impact of pain on daily functions that is experienced. In some embodiments, pain severity is measured on a ten-point scale. In some embodiments, treating primary mitochondrial myopathy with a PPARδ agonist (e.g. Compound 1, or a pharmaceutically acceptable salt thereof) comprises decreases in BPI scores by 1, 2, 3, 4, 5 or more than 5.

In some embodiments, a mammal's fatigue or energy level is evaluated with a Modified Fatigue Impact Scale (MFIS). Fatigue is a feeling of physical tiredness and lack of energy that many people experience from time to time. In some embodiments, people who have medical conditions like primary mitochondrial myopathy experience stronger feelings of fatigue more often and with greater impact than others. MFIS comprises a questionnaire that assesses the impact fatigue has on a person's daily life. In some embodiments, the total MFIS score can range from 0 to 84. In some embodiments, treating primary mitochondrial myopathy with a PPARδ agonist (e.g. Compound 1, or a pharmaceutically acceptable salt thereof) comprises decreases in MFIS scores by 1, 2, 3, 4, 5, 6, 7, 8, 9, 10, 11, 12, 13, 14, 15, 16, 17, 18, 19, 20, or more than 20.

### Combination Treatments

In certain instances, it is appropriate to administer a PPARδ agonist compound (e.g. Compound 1, or a pharmaceutically acceptable salt thereof), in combination with one or more other therapeutic agents.

In one embodiment, the therapeutic effectiveness of a PPARδ agonist compound (e.g. Compound 1), or a pharmaceutically acceptable salt or solvate thereof, is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering a PPARδ agonist compound (e.g. Compound 1), or a pharmaceutically acceptable salt or solvate thereof, with another agent (which also includes a therapeutic regimen) that also has therapeutic benefit.

In one specific embodiment, a PPARδ agonist compound (e.g. Compound 1), or a pharmaceutically acceptable salt or solvate thereof, is co-administered with a second therapeutic agent, wherein a PPARδ agonist compound (e.g. Compound 1), or a pharmaceutically acceptable salt or solvate thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of a PPARδ agonist compound (e.g. Compound 1), or a pharmaceutically acceptable salt or solvate thereof, will be utilized in formulating pharmaceutical composition and/or in treatment regimens when a PPARδ agonist compound (e.g. Compound 1), or a pharmaceutically acceptable salt or solvate thereof, is administered in combination with one or more additional agent, such as an additional therapeutically effective drug, an adjuvant or the like. Therapeutically effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a PPARδ agonist compound (e.g. Compound 1), or a pharmaceutically acceptable salt or solvate thereof, is initiated prior to, during, or after treatment with a second agent described herein, and continues until any time during treatment with the second agent or after termination of treatment with the second agent. It also includes treatments in which a PPARδ agonist compound (e.g. Compound 1), or a pharmaceutically acceptable salt or solvate thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, a PPARδ agonist compound (e.g. Compound 1), or a pharmaceutically acceptable salt or solvate thereof, is administered either simultaneously with the one or more other therapeutic agents, or sequentially.

In combination therapies, the multiple therapeutic agents (one of which is a PPARδ agonist compound (e.g. Compound 1), or a pharmaceutically acceptable salt or solvate thereof) are administered in any order or even simultaneously. If administration is simultaneous, the multiple therapeutic agents are, by way of example only, provided in a single, unified form, or in multiple forms (e.g., as a single pill or as two separate pills).

A PPARδ agonist compound (e.g. Compound 1), or a pharmaceutically acceptable salt or solvate thereof, as well as combination therapies, are administered before, during or after the occurrence of a disease or condition, and the timing of administering the composition containing a PPARδ agonist compound (e.g. Compound 1), or a pharmaceutically acceptable salt or solvate thereof, varies. Thus, in one embodiment, Compound I, or a pharmaceutically acceptable salt or solvate thereof, is used as a prophylactic and are administered continuously to subjects with a propensity to develop conditions or diseases in order to prevent the occurrence of the disease or condition. In another embodiment, a PPARδ agonist compound (e.g. Compound 1), or a pharmaceutically acceptable salt or solvate thereof, is administered to a subject during or as soon as possible after the onset of the symptoms. In specific embodiments, a PPARδ agonist compound (e.g. Compound 1), or a pharmaceutically acceptable salt or solvate thereof, is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a PPARδ agonist compound (e.g. Compound 1), or a pharmaceutically acceptable salt or solvate thereof, or a formulation containing Compound I, or a pharmaceutically acceptable salt or solvate thereof, is administered for at least 2 weeks, about 1 month to about 5 years.

### Exemplary Agents for Use in Combination Therapy

In some embodiments, a PPARδ agonist compound (e.g. Compound 1, or a pharmaceutically acceptable salt), is administered in combination with one or more additional therapies used for treating primary mitochondrial myopathy.

In certain embodiments, the at least one additional therapy is administered at the same time as a PPARδ agonist compound (e.g. Compound 1), or a pharmaceutically acceptable salt or solvate thereof. In certain embodiments, the at least one additional therapy is administered less frequently than a PPARδ agonist compound (e.g. Compound 1), or a pharmaceutically acceptable salt or solvate thereof. In certain embodiments, the at least one additional therapy is administered more frequently than a PPARδ agonist compound (e.g. Compound 1), or a pharmaceutically acceptable salt or solvate thereof. In certain embodiments, the at least one additional therapy is administered prior to administration of a PPARδ agonist compound (e.g. Compound 1), or a pharmaceutically acceptable salt or solvate thereof. In certain embodiments, the at least one additional therapy is administered after administration of a PPARδ agonist compound (e.g. Compound 1), or a pharmaceutically acceptable salt or solvate thereof.

In some embodiments, a PPARδ agonist compound (e.g. Compound 1, or a pharmaceutically acceptable salt), is administered in combination with ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, acid, resveratrol, N-acetyl-L-cysteine (NAC), zinc, folinic acid/leucovorin calcium, or a combination thereof.

In some embodiments, a PPARδ agonist compound (e.g. Compound 1, or a pharmaceutically acceptable salt), is administered in combination with ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, KH176, or a combination thereof

In some embodiments, a PPARδ agonist compound (e.g. Compound 1, or a pharmaceutically acceptable salt), is administered in combination with succinic acid, or salt thereof, or trisuccinylglycerol, or salt thereof. In some embodiments, a PPARδ agonist (e.g. Compound 1, or a pharmaceutically acceptable salt), is administered in combination with a compound described in International PCT publication no. WO 2017/184583.

In some embodiments, a PPARδ agonist compound (e.g. Compound 1, or a pharmaceutically acceptable salt), is administered in combination with an antioxidant.

In some embodiments, a PPARδ agonist compound (e.g. Compound 1, or a pharmaceutically acceptable salt), is administered in combination with an odd-chain fatty acid, odd-chain fatty ketone, L-carnitine, or combinations thereof.

In some embodiments, a PPARδ agonist compound (e.g. Compound 1, or a pharmaceutically acceptable salt), is administered in combination with triheptanoin, n-heptanoic acid, a triglyceride, or a salt or thereof, or combinations thereof.

In some embodiments, a PPARδ agonist compound is administered in combination with a Nicotinamide Adenine Dinucleotide (NAD+) pathway modulator. NAD+ plays many important roles within cells, including serving as an oxidizing agent in oxidative phosphorylation which generates ATP from ADP. Increasing cellular concentrations of NAD+ will enhance the oxidative capacity within mitochondria, thereby increasing nutrient oxidation and boost energy supply, which is a primary role of mitochondria. In some embodiments the NAD+ modulator targets Poly ADP Ribose Polymerase (PARP), Aminocarboxymuconate Semialdehyde Decarboxylase (ACMSD) and N′-Nicotinamide Methyltransferase (NNMT).

### Kits and Articles of Manufacture

Described herein are kits for treating treatment of primary mitochondrial myopathy in an individual comprising administering to said individual a PPARδ agonist compound (e.g. Compound 1, or a pharmaceutically acceptable salt thereof).

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any treatment of primary mitochondrial myopathy) that benefits from PPARδ modulation.

The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In some embodiments, a label is on or associated with the container. A label, in some cases, is on a container when letters, numbers or other characters forming the label are attached, molded or etched into the container itself; a label, in some cases, is associated with a container when it is present within a receptacle or carrier that also holds the container, e.g., as a package insert. A label, in some cases, is used to indicate that the contents are to be used for a specific therapeutic application. The label, in some cases, indicates directions for use of the contents, such as in the methods described herein.

In certain embodiments, a pharmaceutical composition comprising a PPARδ agonist compound (e.g. Compound 1, or a pharmaceutically acceptable salt thereof), is presented in a pack or dispenser device which, in some cases, contains one or more unit dosage forms. The pack, in some cases, for example contains metal or plastic foil, such as a blister pack. The pack or dispenser device, in some cases, is accompanied by instructions for administration.

The pack or dispenser, in some cases, is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, in some cases, is the labeling approved by the U.S. Food and Drug Administration for prescription drugs, or the approved product insert. Compositions containing a compound provided herein formulated in a compatible pharmaceutical carrier, in some cases, is also prepared, placed in an appropriate container, and labeled for treatment of an indicated condition.

### EXAMPLES

The following examples are provided for illustrative purposes only and not to limit the scope of the claims provided herein.

### Example 1: Cell Lines and Culture

Subjects. Skin biopsies for fibroblast culture are performed on a clinical basis with written informed consent from subjects and/or legal guardians. Fibroblast cells with mutations in any one of the genes and/or proteins associated with a primary mitochondrial myopathy are obtained from patients' skin biopsies, while wild type (WT) fibroblast cells are obtained from healthy individuals.

In some embodiments, fibroblast cells are obtained from subjects with a confirmed diagnosis of a primary mitochondrial myopathy (e.g., m.3243A>G mutation or mtDNA mutations) or they are purchased is available from commercial sources, e.g. from the Coriell Institute Coriell Institute for Medical Research (403 Haddon Avenue, Camden, N.J. 08103).

Cell culture and treatments. Cells are grown in Dulbecco's Modified Eagle Medium (DMEM), Corning Life Sciences, Manassas, Va., containing high glucose levels or in DMEM devoid of glucose for 48-72 hr. Both media are supplemented with fetal bovine serum, glutamine, penicillin and/or streptomycin. In some experiments, fibroblasts are incubated with N-acetylcysteine, resveratrol, mitoQ, Trolox (a hydro-soluble analogue of vitamin E), or bezafibrate, prior to the analysis of parameters.

A PPARδ agonist compound is dissolved in phosphate buffer saline, PBS, as a stock solution. Amounts are added appropriately directly to cell culture media in flasks when the cultures are about 85-90% confluent. The cultures are allowed to grow for 48 h at 37° C., and then harvested. Harvested cell pellets are stored at −80° C. until immune and enzymatic assays analyses. 1 mL to 1.5 mL media samples are also stored at −80° C. for acylcarnitines.

### Example 2: Measurement of Mitochondrial Respiration

Oxygen consumption rate (OCR) is measured with a Seahorse XFe96 Extracellular Flux Analyzer (Sea horse Bioscience, Billerica, Mass.).

Briefly, the apparatus contains a fluoro-phore that is sensitive to changes in oxygen concentration, which enables it to accurately measure the rate at which cytochrome c oxidase (complex IV) reduces one O₂ molecule to two H₂O molecules during OXPHOS. Cells are seeded in 96-well Seahorse tissue culture microplates in growth media at a density of 80,000 cells per well. To ensure equal cell numbers, cells are seeded in cell culture plates pre-coated with Cell-Tak, BD Biosciences, San Jose, Calif. All cell lines are measured with four to eight wells per cell line. Then, the entire set of experiments is repeated. Before running the Seahorse assay, cells are incubated for 1 hour without CO₂ in unbuffered DMEM. Initial OCR is measured to establish a baseline (basal respiration). Maximal respiration is also determined after the injection of 300 nM carbonyl cyanide 4-(trifluoromethoxy)phenylhydrazone (FCCP), Seahorse XF Cell Mito Stress Test Kit, Santa Clara, Calif.

### Example 3: ATP Production Assay

ATP production is determined by a bioluminescence assay using an ATP determination kit (ATPlite kit) from PerkinElmer Inc, Waltham, Mass., according to the manufacturer's instructions.

### Example 4: Fatty Acid Oxidation (FAO) Flux Analysis

Fatty acid oxidation (FAO) flux analysis is performed by quantifying the production of ³H₂O from 9,10-[³H]palmitate, PerkinElmer, Waltham, Mass., conjugated to fatty acid-free albumin in fibroblasts cultured in a 24-well plate.

A representative non-limiting example of a FAO flux analysis is described in Bennett, M. J. Assays of fatty acid beta-oxidation activity. Methods Cell Biol 80, 179-197, (2007)). In some embodiments, 300,000 fibroblasts are plated per well in 6-well plates and grown for 24 hr in DMEM with 10% fetal bovine serum. The growth media is then changed to either the same media or devoid of glucose and fibroblasts are grown as described for 48 hours. Subsequently, cells are washed once with PBS and then incubated with 0.34 μCi [9,10-³H]oleate (45.5 Ci/mmol; Perkin Elmer, Waltham, Mass.) in 50 nmol of oleate prepared in 0.5 mL glucose-free DMEM with 1 μ/ml carnitine and 2 mg/ml α-cyclodextrin for 2 hours at 37° C. Fatty acids are solubilized with α-cyclodextrin as described (Watkins, P. A., Ferrell, E. V. Jr., Pedersen, J. I. & Hoefler, G. Peroxisomal fatty acid beta-oxidation in HepG2 cells. Arch Biochem Biophys 289, 329-336 (1991)). After incubation, ³H₂O released is separated from the oleate on a column containing 750 μL of anion exchange resin (AG 1×8, acetate, 100-200 Mesh, BioRad, Richmond, Calif.) prepared in water. After the incubation medium passes through the column, the plate is washed with 750 μL of water which is also transferred to the column. The resin is then washed twice with 750 μL of water. All eluates are collected in a scintillation vial and mixed with 5 mL of scintillation fluid (Eco-lite, MP), followed by counting in a Beckman scintillation counter in the tritium window. Assays are performed in quadruplicate with triplicate blanks (cell free wells). Standards contain a 50 μL aliquot of the incubation mix with 2.75 mL of deionized water and 5 mL of scintillation fluid.

### Example 5: Western Blotting

Cells are grown in T175 flasks and, at 90-95% confluence, are harvested by trypsinization, pelleted and stored at −80° C. for western blot. Protein content in samples is quantified for data normalization using DC™ Protein Assay kit (Bio-Rad Laboratories).

For cell lysates, pellets are re-suspended in 150-250 μL of RIPA buffer with protease inhibitor cocktail, Roche Diagnostics, Mannheim, Germany. Homogenates are kept on ice for 30 minutes, shaken every 10 minutes, and centrifuged. Supernatants are used for western blotting. For mitochondria, pellets are re-suspended in 150-250 μL of 5 mM Tris buffer, pH 7.4, containing 250 mM sucrose, 2 mM EDTA, protease inhibitor cocktail, Roche Diagnostics, Mannheim, Germany, and 0.5 μM trichostatin A, Sigma-Aldrich Co., St. Louis, Mo., homogenized and centrifuged. The pellet is discarded and the supernatant centrifuged. The resulting pellet containing mitochondria is re-suspended in 50 mM Tris buffer, pH 7.4, sonicated and centrifuged again.

Cell lysates or mitochondria are used for western blotting as previously described (e.g. Goetzman, E. S. et al. Mol. Genet. Metab. 91, 138-147, (2007)).

### Example 6: Immunofluorescence Microscopy and Mitochondrial Membrane Potential (ΔΨ)

Cells are incubated with the antibodies anti-VLCAD (1:1000), anti-Nrf2 (1:100) or anti-NF-kB (1:1000) at 4° C. overnight. After brief washing with TBST, cells are incubated with donkey anti-rabbit secondary antibody Alexa Fluor 488, from Invitrogen. Nuclei are immunostained with DAPI. The coverslips are then mounted using mounting media before taking images with an Olympus Confocal FluoroView1000 microscope at a magnification of 60×.

### Example 7: Cell Viability Assay

Cell viability is evaluated with a 3-(4,5-dimethylthiazol-2-yl)-5-(3-carboxymethoxyphenyl)-2-(4-sulfophenyl)-2H-tetrazolium (MTS) assay kit according to the manufacturer's instructions, Abcam, Cambridge, Mass. The absorbance is read in the FLUOstar Omega plate reader at 490 nm.

### Example 8: Apoptosis Assay

Apoptosis is evaluated with an Alexa Fluor® 488 annexin V/Dead Cell Apoptosis kit according to manufacturer's instructions, Invitrogen, Grand Island, N.Y. The kit contains annexin V labeled with a fluorophore and propidium iodide (PI). Annexin V can identify apoptotic cells by binding to phosphatidylserine exposed on the outer leaflet of cell plasma membrane while PI stains dead cells by binding to nucleic acids. Fluorescence is determined in a Becton Dickinson FACSAria II flow cytometer, BD Biosciences, San Jose, Calif.

### Example 9: Determination of Acylcarnitine Levels

Acylcarnitine analysis is performed utilizing the appropriate tandem mass spectrometry (MS/MS) protocols.

### Example 10: In Vivo Gene Expression Evaluation in Mouse Muscle

Male C57BL/6 mice were administered an oral dose of Compound 1 at 30 mg/kg, once daily for 7 consecutive days. Four hours following the final dose administration on day 7, all mice were euthanized, and two samples of quadriceps muscle were dissected from the right and left limbs. Compound 1 treatment altered the expression patterns of several well-known PPARδ regulated genes and pathways important for fatty acid transport into mitochondria (CPT1b), oxidative phosphorylation (PDK4) and mitochondrial biogenesis (PGC-1α).

In a second study, male C57BL/6 mice were dosed once daily for four consecutive days. On the first day of treatment all mice in each group received a single dose of either the vehicle or Compound 1 at 30 mg/kg. Following the dose administration on the first day, five mice from each group were anesthetized and euthanized at each of the following time points: 1, 2, 4, 8, 24, 48, 72 and 96 hours. Animals left after the time point 24 hours received the second dose. Animals left after the time point 48 hours received the third dose. Animals left after the time point 72 hours received the fourth dose. Mice designated for time point 96 hours were euthanized on day 5. At 48 hrs Compound 1 treatment increased the expression of PGC1□, the master regulator of mitochondrial biogenesis, and CPT1b, the rate-controlling enzyme of the long-chain fatty acid beta-oxidation pathway in muscle mitochondria.

### Example 11: Combination Therapy

In some embodiments, PPARδ agonists are used in combination with other therapies for primary mitochondrial myopathy (PMM). In some embodiments, a PPARδ agonist compound is administered to an individual with a (PMM) in combination with one or more of the following: ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, and KH176.

Combination therapy is advantageous when efficacy is greater than either agent alone or when the dose required for either drug is reduced thereby improving the side effect profile.

### Example 12: Determination of Binding Selectivity of Compound 1 to PPARα, PPARδ, and PPARδ

Compound 1 was tested on all three human PPAR subtypes (hPPAR): hPPARα, hPPARγ, and hPPARδ. The results of representative experiments in each human PPAR subtype are shown in Table 1. All assays were repeated at least three times for each subtype. Compound 1 is a potent and efficacious agonist of PPARδ, (EC50=31 nM), whereas the compound only shows minor activity on PPARα (EC50>10 μM) and PPARγ (EC50>10 M).

The genes of interest were synthesized and cloned into an appropriate Jump-In™ retargeting vector following the User Guide of the Jump-In™ T-REx™ HEK293 Retargeting Kit (ThermoFisher Catalog No A15008). For example, the vector will be used to transfect and retarget the Jump-In™ HEK293 GripTite™ parental cell line. Stable pools will be antibiotic-selected for about 21 days and tested for target gene expression by functional assay.

### Retargeting Methods:

Jump-In™ GripTite™ HEK293 parental cells were plated at 60-80% confluency in a T-75 flask in growth medium without antibiotics and transfected with a 1:1 ratio of expression construct and R4 integrase expression construct (20 μg DNA total) using Lipofectamine® LTX (50 μL) and PLUS™ Reagent (20 μL). Following a 48 hours incubation, cells were selected with 600 μg/mL Geneticin® and 10 μg/mL Blasticidin for ˜21 days in growth medium.

### BLA Assay Methods:

Jump-In™ GripTite™ HEK293 rPPAR Alpha, Delta or Gamma UAS-bla-Gal4 cell pools were plated in a 384-well plate format (20,000 cells per well) in OptiMeM without FBS in replicates (n=4). Cells were allowed to adhere for 8 hours before addition of Compound 1 (1 mM top concentration, 3-fold dilutions, 10-point titration). After 16 hours, the cells were loaded with LiveBLAzer®, a fluorescent BLA substrate that gives a blue/green readout of expressing/non-expressing cells, respectively. The blue/green readout was measured on a fluorescent plate reader (Tecan Safire II).

### Example 13: Clinical Trial for Primary Mitochondrial Myopathy (PMM)

A non-limiting example of a primary mitochondrial myopathy clinical trial in humans is described below.

Purpose: The purposes of this study was: to assess the safety and tolerability of 12 weeks treatment with Compound 1, or a pharmaceutically acceptable salt or solvate thereof, in subjects with primary mitochondrial myopathy; to investigate pharmacokinetics of Compound 1, or a pharmaceutically acceptable salt or solvate thereof, in subjects with primary mitochondrial myopathy treated with Compound 1, or a pharmaceutically acceptable salt or solvate thereof; to investigate the pharmacodynamics effects of Compound 1, or a pharmaceutically acceptable salt or solvate thereof, in subjects with primary mitochondrial myopathy treated with Compound 1, or a pharmaceutically acceptable salt or solvate thereof.

Intervention: Patients were administered 10-2000 mg of Compound 1, or a pharmaceutically acceptable salt or solvate thereof, per day as single agent or in combination. For example, subjects received 100 mg of Compound 1, or a pharmaceutically acceptable salt or solvate thereof, once daily for a total of 12 weeks. Other cohorts are contemplated.

Compound 1, or a pharmaceutically acceptable salt or solvate thereof, will be packed in bottles as capsules.

Detailed Description: Patients were given Compound 1, or a pharmaceutically acceptable salt or solvate thereof, orally once a day.

Inclusion Criteria: Primary mitochondrial myopathy (PMM) as defined by the International Workshop: Outcome measures and clinical trial readiness in primary mitochondrial myopathies in children and adults (Mancuso, M. et al. (2017, December). International Workshop: Outcome measures and clinical trial readiness in primary mitochondrial myopathies in children and adults. Consensus recommendations. 10-18 Nov. 2016, Rome, Italy. Neuromuscul. Disord., 12, 1126-1137), and with a myopathy score of 2-4 on the Newcastle Mitochondrial Disease Adult Scale (NMDAS) Section III Question 5. Approximately 12 subjects have a confirmed m.3243A>G mutation and 12 subjects have other mtDNA defects, with myopathy.

Currently following a stable dietary regimen with avoidance of fasting as documented by a 3-day dietary record obtained during the screening period.

A stable treatment regimen for at least 30 days prior to enrollment.

Expected and willing to remain on stable diet and medication through the study.

Ambulatory and able to perform the study exercise tests.

Adequate kidney function defined as an estimated glomerular filtration rate (eGFR)≥60 mL/min/1.73 m2 using the Cockcroft-Gault formula.

Able to swallow capsules.

Exclusion Criteria: Subjects presenting with any of the following will not be included in the study:
unstable or poorly controlled disease as determined by one or more of the following: echocardiogram with evidence of active or worsening cardiomyopathy at screening; presence of symptoms of acute rhabdomyolysis with elevations in serum CPK consistent with acute exacerbation of myopathy; evidence of acute crisis from their underlying disease.
currently taking anticoagulants.
have motor abnormalities other than those related to the mitochondrial disease that could interfere with the outcome measures.
treatment with an investigational drug within 3 months prior to Day 1.
evidence of significant concomitant clinical disease that in the opinion of the Investigator may need a change in management during the study or could interfere with the conduct or safety of this study. (Stable well-controlled chronic conditions such as controlled hypertension (BP<140/90 mmHg) thyroid disease, well-controlled Type 1 or Type 2 diabetes (HbA1c<8%), hypercholesterolemia, gastroesophageal reflux, or depression under control with medication (other than tricyclic antidepressants), are acceptable provided the symptoms and medications would not be predicted to compromise safety or interfere with the tests and interpretations of this study).
history of cancer with the exception of in situ skin cancer.
have been hospitalized within the 3 months prior to screening for any major medical condition (as deemed by the primary investigator).
clinically significant cardiac disease or ECG abnormalities.
any condition possibly reducing drug absorption (e.g., gastrectomy).
history of clinically significant liver disease as evidenced by elevations in ALT, GGT or TB.
positive hepatitis B surface antigen (HBsAg) or hepatitis C, or HIV at screening.
evidence of clinically significant muscle damage tests (CPK>3×ULN)
history of drug abuse or with a positive urine drug screen.
history of regular alcohol consumption exceeding 14 drinks/week (1 drink=150 mL of wine or 360 mL of beer or 45 mL of spirits) within 6 months of screening.
pregnant or nursing females.
history of sensitivity to PPAR agonists.
any other severe acute or chronic medical or psychiatric condition or laboratory abnormality that in the opinion of the Investigator may increase the risk associated with study participation or investigational product administration or may interfere with the interpretation of study results.

Outcome Measures: Safety Endpoints include: number and severity of adverse events. Absolute values, changes from baseline at Week 12 and incidence of clinically significant changes in: laboratory safety tests; electrocardiograms; supine vital signs.

Pharmacokinetic Endpoints include: Compound 1 plasma concentrations and identification of metabolites using pooled plasma.

Absolute values and changes from baseline to Week 12 in serum biomarkers: fibroblast growth factor 21 (FGF-21) and growth/differentiation factor 15 (GDF-15). Absolute values and changes from baseline to Week 12 in acylcarnitine panel. Changes from baseline to Week 12 in muscle histopathology.

Changes from baseline following 12 weeks of treatment with Compound 1 in: peak exercise test (including Borg scale); sub-maximal exercise test (including Borg scale); distance walked during a 12-minute walk test (including gait analysis); and 30 second sit to stand.

Change from baseline following 12 weeks of treatment with Compound 1 in muscle biopsy biomarkers (in order of importance if sample is sparse): mitochondrial DNA copy number; heteroplasmy level, respiratory chain enzyme activity (ATP-ADP levels, fatty acid oxidation gene expression or flux); messenger ribonucleic acid (mRNA) levels using transcriptomics; change from baseline in NMDAS; change from baseline in the SF-36; change from baseline in the Modified Fatigue Impact Scale score; change from baseline in Brief Pain Inventory (short form).

PMM Clinical Trial Results with Compound 1

In general, Compound 1 was well tolerated among subjects that participated in the study.

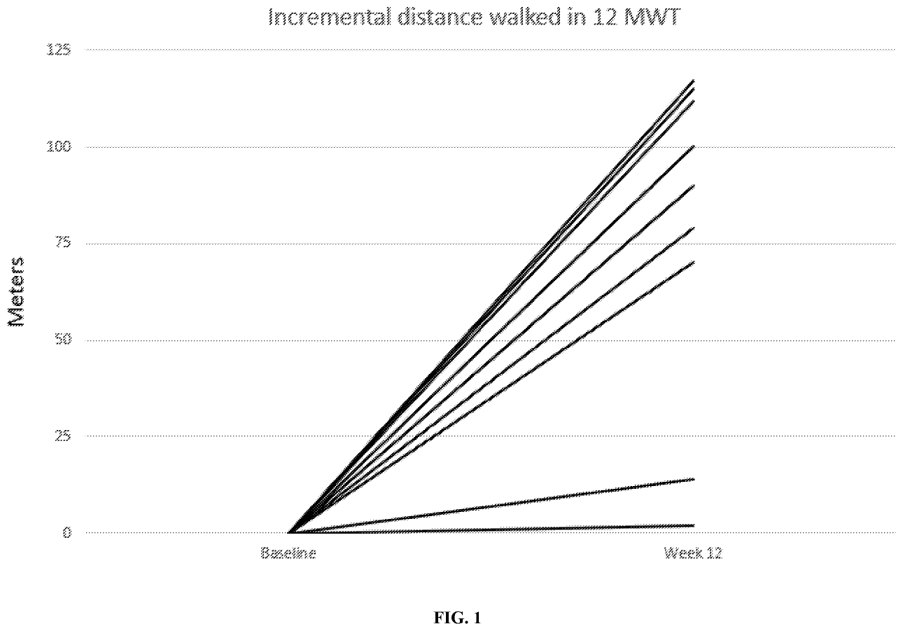

Improvements in exercise tolerance was observed in subjects that received 100 mg of Compound 1, or a pharmaceutically acceptable salt or solvate thereof, once daily for a total of 12 weeks. Subjects were able to increase the distance walked during a 12-minute walk test. FIG. 1 shows the results of the impact of Compound 1 on the 12-minute walk test in this group of subjects. In this same group of subjects, trends towards increases in peak VO₂ were observed for many subjects that received 100 mg of Compound 1, or a pharmaceutically acceptable salt or solvate thereof, once daily for a total of 12 weeks.

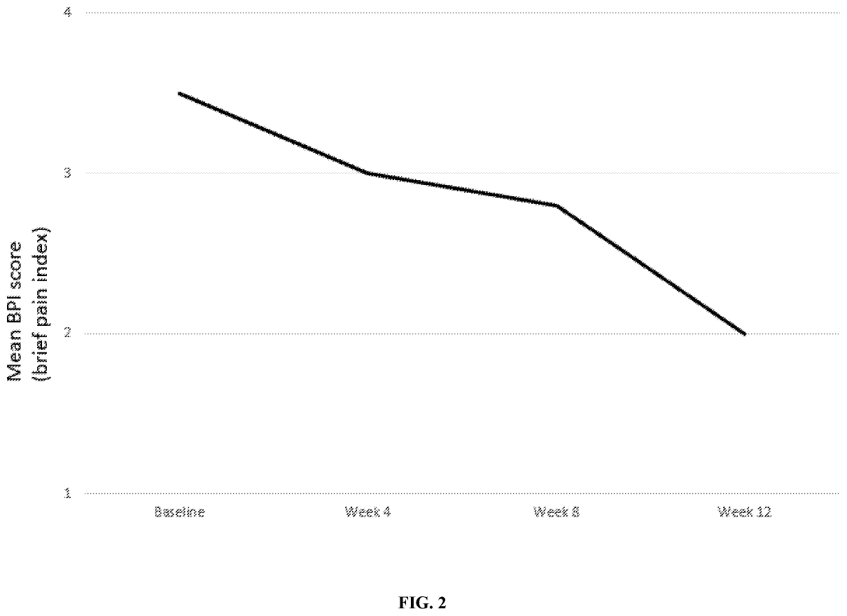

Decreases in the brief pain index (BPI) was observed in subjects that received 100 mg of Compound 1, or a pharmaceutically acceptable salt or solvate thereof, once daily for a total of 12 weeks. FIG. 2 shows the decreases in the mean BPI scores resulting from administration of Compound 1, or a pharmaceutically acceptable salt or solvate thereof, to this group of subjects. In this same group of subjects, trends towards increases in Modified Fatigue Impact Scale scores were observed for many subjects that received 100 mg of Compound 1, or a pharmaceutically acceptable salt or solvate thereof, once daily for a total of 12 weeks.

The examples and embodiments described herein are for illustrative purposes only and various modifications or changes suggested to persons skilled in the art are to be included within the spirit and purview of this application and scope of the appended claims.

What is claimed is:

1. A method for treating a primary mitochondrial myopathy in a mammal comprising administering to the mammal with a primary mitochondrial myopathy a peroxisome proliferator-activated receptor delta (PPARδ) agonist compound.

2. The method of claim 1, wherein:
treating the primary mitochondrial myopathy comprises increasing oxidative phosphorylation (OXPHOS) in the mammal, improving the mammal's exercise tolerance, improving muscle histology, improving mitochondrial DNA copy number, improving heteroplasmy levels, improving the quality of mitochondria, decreasing pain, decreasing fatigue, improving cognition, improving overall well-being, increasing survival or a combination thereof.

3. The method of claim 1 or claim 2, wherein:
the PPARδ agonist compound is administered to the mammal in an amount sufficient for increasing OXPHOS capacities in the mammal, up-regulating gene expression of any one of the enzymes or proteins involved in OXPHOS, or a combination thereof.

4. The method of any one of claims 1-3, wherein:
the PPARδ agonist compound is administered to the mammal in an amount sufficient for increasing fatty acid oxidation (FAO) capacities in the mammal, up-regulating gene expression of any one of the enzymes or proteins involved in FAO, or a combination thereof.

5. The method of any one of claims 1-4, wherein the mammal with a primary mitochondrial myopathy has:
at least one mutation or deletion in at least one mitochondrial DNA (mtDNA) gene;
at least one mitochondrial DNA (mtDNA) defect;
at least one mutation or deletion in at least one nuclear DNA (nDNA) gene involved in mitochondrial function; or
a combination thereof.

6. The method of claim 5, wherein:
the at least one mutation in at least one mitochondrial DNA (mtDNA) gene comprises a mutation selected from m.3243A>G, m.8344A>G, m.8993T>G, m.13513G>A, m.11778G>A, m.14484T>C, and a combination thereof.

7. The method of claim 5, wherein:
the at least one mutation in at least one mitochondrial DNA (mtDNA) gene comprises a mutation selected from a 8284 bp deletion, a 6277 bp deletion, a 4977 bp deletion, and a combination thereof.

8. The method of claim 5, wherein:
the at least one mutation or deletion in at least one nuclear DNA (nDNA) gene involved in mitochondrial function comprises at least one mutation or deletion in a nDNA gene encoding complex I (NADH:ubiquinone oxidoreductase), complex II (succinate dehydrogenase), complex III (CoQ-cytochrome c reductase), complex IV (cytochrome c oxidase), complex V (ATP synthase), an aminoacyl-tRNA synthetase, a release factor, an elongation factor, a mitoribosomal protein, solute carriers of thiamine and phosphate, or a combination thereof.

9. The method of claim 8, wherein:
the gene encoding the complex I comprises NDUFS1, NDUFS2, NDUFS3, NDUFS4, NDUFS6, NDUFS7, NDUFS8, NDUFVJ, NDUFV2, NDUFA1, NDUFA2, NDUFA9, NDUFA10, NDUFA11, NDUFA12, NDUFA13, NDUFAF2, NDUFAF6, or NDUFB11;
the gene encoding the complex II comprises SDHA, SDHB, SDHC, SDHD, or SDHAF1;
the gene encoding the complex III comprises UQCRB, BCS1L, UQCRQ, UQCRC2, CYC1, TTC19, LYRM7, UQCC2, or UQCC3;
the gene encoding the complex IV comprises COA5, SURF1, COX10, COX14, COX15, COX20, COX6B1, FASTKD2, SCO1, SCO2, LRPPRC, TACO1, or PET100;
the gene encoding the complex V comprises ATPAF2, TMEM70, ATP5E, or ATP5A1;
the gene encoding the aminoacyl-tRNA synthetase comprises AARS2, DARS2, EARS2, RARS2, YARS2, FARS2, HARS2, LARS2, VARS2, TARS2, IARS2, CARS2, PARS2, NARS2, KARS, GARS, SARS2, or M4RS2;
the gene encoding the release factor comprises C12orf65;
the gene encoding the elongation factor comprises TUFM, TSFM, or GFM1;
the gene encoding the mitoribosomal protein comprises MRPS16, MRPS22, MRPL3, MRP12, or MRPL44; and
the gene encoding the solute carriers of thiamine and phosphate comprises SLC19A3, SLC25A3, or SLC25A19.

10. The method of claim 5, wherein:
the at least one mutation or deletion in at least one nuclear DNA (nDNA) gene involved in mitochondrial function comprises at least one mutation or deletion in a nDNA gene involved in phospholipid metabolism, metabolism of toxic compounds, disulfide relay system, iron-sulfur protein assembly, tRNA modification, mRNA processing, mitochondrial fusion or fission, deoxynucleotide triphosphate synthesis, protein quality control and degradation, ATP and ADP transport, or a combination thereof.

11. The method of claim 10, wherein:
the gene involved in the phospholipid metabolism comprises AGK, SERAC1, or TAZ;
the gene involved in the metabolism of toxic compounds comprises HIBCH, ECHS1, ETHE1, or MPV17;
the gene involved in the disulfide relay system comprises GFER;
the gene involved in the iron-sulfur protein assembly comprises ISCU, BOLA3, NFU1, or IBA57;
the gene involved in the tRNA modification comprises MTO1, GTP3BP, TRMU, PUS1, MTFMT, TRIT1, TRNT1, or TRMT5;
the gene involved in the mRNA processing comprises LRPPRC, TACO1, ELAC2, PNPT1, HSD17B10, MTPAP, or PTCD1;
the gene involved in the mitochondrial fusion and fission comprises OPA1 or MFN2;
the gene involved in the deoxynucleotide triphosphate synthesis comprises DGUOK, TK2, TYMP, MGME1, SUCLG1, SUCLA2, RNASEH1, C10orf2, POLG, POLG2, DNA2, or RRM2B;
the gene involved in the protein quality control and degradation comprises FBXL4, AFG3L2, or SPG7; and
the gene involved the ATP and ADP transport comprises ANT1.

12. The method of any one of claims 1 to 11, wherein:
the mammal has been diagnosed with Kearns-Sayre syndrome (KSS), Leigh syndrome, maternally inherited Leigh syndrome (MILS), Mitochondrial DNA depletion syndrome (MDS), Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy ataxia and retinitis pigmentosa (NARP), Pearson syndrome, or Progressive external ophthalmoplegia (PEO).

13. The method of any one of claims 1-12, wherein:
the mammal with a primary mitochondrial myopathy also comprises a secondary mitochondrial myopathy.

14. The method of claim 13, wherein:
wherein the secondary mitochondrial myopathy involves secondary defects in OXPHOS function due to primary FAO deficiencies, or the secondary mitochondrial myopathy results from a primary OXPHOS deficiency that results in secondary FAO disease.

15. The method of any one of claims 1-14, wherein the PPARδ agonist compound increases mitochondrial biogenesis.

16. The method of any one of claims 1-15, wherein the PPARδ agonist compound increases expression or activity of a gene or protein involved in mitochondrial biogenesis.

17. The method of claim 16, wherein the protein is peroxisome proliferator-activated receptor gamma coactivator 1-alpha (PGC-1α).

18. The method of any one of claims 1-17, wherein, the PPARδ agonist compound increases expression or activity of a gene or protein thereof involved in oxidative phosphorylation.

19. The method of any one of claims 1-18, wherein, the PPARδ agonist compound increases the percentage of non-mutated mitochondrial DNA (mtDNA) relative to the proportion of mutated mtDNA.

20. The method of any one of claims 1-19, wherein:
the PPARδ agonist compound binds to and activates the cellular PPARδ and does not substantially activate the cellular peroxisome proliferator activated receptors alpha (PPARα) and gamma (PPARγ).

21. The method of any one of claims 1-20, wherein:
the PPARδ agonist compound is a phenoxyalkylcarboxylic acid compound.

22. The method of any one of claims 1-21, wherein:
the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound.

23. The method of any one of claims 1-21, wherein:
the PPARδ agonist compound is a phenoxyethanoic acid compound or a phenoxyhexanoic acid compound.

24. The method of claim 21, wherein:
the PPARδ agonist compound is an allyloxyphenoxyethanoic acid compound.

25. The method of any one of claims 1-19, wherein the PPARδ agonist compound is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid; or
{4-[3,3-Bis-(4-bromo-phenyl)-allyloxy]-2-methyl-phenoxy}-acetic acid;
or a pharmaceutically acceptable salt thereof.

26. The method of any one of claims 1-20, wherein the PPARδ agonist compound is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid;
{4-[3,3-Bis-(4-bromo-phenyl)-allyloxy]-2-methyl-phenoxy}-acetic acid;
(R)-3-methyl-6-(2-((5-methyl-2-(4-(trifluoromethyl)phenyl)-1H-imidazol-1-yl)methyl)phenoxy)hexanoic acid;
(R)-3-methyl-6-(2-((5-methyl-2-(6-(trifluoromethyl)pyridin-3-yl)-1H-imidazol-1-yl)methyl)phenoxy)hexanoic acid;
2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954);
2-[2-methyl-4-[[3-methyl-4-[[4-(trifluoromethyl)phenyl]methoxy]phenyl]thio]phenoxy]-acetic acid;
2-[2-methyl-4-[[[4-methyl-2-[4-(trifluoromethyl)phenyl]-5-thiazolyl]methyl]thio]phenoxy]-acetic acid (GW-501516);
2-[[[2-[3-Fluoro-4-(trifluoromethyl)phenyl]-4-methyl-5-thiazolyl]methyl]thio]-2-methylphenoxy]acetic acid (GW0742 also known as GW610742);
2-[2,6 dimethyl-4-[3-[4-(methylthio)phenyl]-3-oxo-1(E)-propenyl]phenoxyl]-2-methylpropanoic acid (elafibranor; GFT-505);
{2-methyl-4-[5-methyl-2-(4-trifluoromethyl-phenyl)-2H-[1,2,3]triazol-4-ylmethylsulfanyl]-phenoxy}-acetic acid;
2-({(2R)-2-Ethoxy-3-[4-(trifluoromethyl)phenoxy]propyl}sulfanyl)-2-methylphenoxy]acetic acid (seladelpar; MBX-8025);
(S)-4-[cis-2,6-dimethyl-4-(4-trifluoromethoxy-phenyl)piperazine-1-sulfonyl]-indan-2-carboxylic acid or a tosylate salt thereof (KD-3010);
(2s)-2-{4-butoxy-3-[({[2-Fluoro-4-(Trifluoromethyl)phenyl]carbonyl}amino)methyl]benzyl}butanoic acid (TIPP-204);
2-[3-(4-Acetyl-3-hydroxy-2-propylphenoxy)propoxy]phenoxy]acetic acid (L-165,0411);
2-(4-{2-[(4-Chlorobenzoyl)amino]ethyl}phenoxy)-2-methylpropanoic acid (bezafibrate);
2-(2-methyl-4-(((2-(4-(trifluoromethyl)phenyl)-2H-1,2,3-triazol-4-yl)methyl)thio)phenoxy)acetic acid; or
(R)-2-(4-((2-ethoxy-3-(4-(trifluoromethyl)phenoxy)propyl)thio)phenoxy)acetic acid;
or a pharmaceutically acceptable salt thereof.

27. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof.

28. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg.

29. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg.

30. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 75 mg to about 125 mg.

31. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg.

32. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 100 mg.

33. The method of any one of claims 1-32, wherein:
the PPARδ agonist compound is systemically administered to the mammal.

34. The method of claim 33, wherein:
the PPARδ agonist compound is administered to the mammal in the form of an oral solution, oral suspension, powder, pill, tablet or capsule.

35. The method of any one of claims 1-34, wherein:
the PPARδ agonist compound is administered to the mammal daily.

36. The method of any one of claims 1-34, wherein:
the PPARδ agonist compound is administered to the mammal once daily.

37. The method of any one of claims 1-35, further comprising:
administering at least one additional therapeutic to the mammal.

38. The method of claim 37, wherein:
the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, KH176, or a combination thereof.

39. The method of claim 37, wherein:
the at least one additional therapeutic is an odd-chain fatty acid, odd-chain fatty ketone, L-carnitine, or combinations thereof.

40. The method of claim 37, wherein:
the at least one additional therapeutic is triheptanoin, n-heptanoic acid, a triglyceride, or a salt or thereof, or combinations thereof.

41. The method of any one of claims 1-40, wherein the mammal is a human.

42. A method for treating a primary mitochondrial myopathy in a mammal comprising administering to the mammal with a primary mitochondrial myopathy a PPARδ agonist compound, wherein the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof.

43. The method of claim 42, wherein:
treating the primary mitochondrial myopathy comprises increasing oxidative phosphorylation (OXPHOS) in the mammal, improving the mammal's exercise tolerance, improving muscle histology, improving mitochondrial DNA copy number, improving heteroplasmy levels, improving the quality of mitochondria, decreasing pain, decreasing fatigue, improving cognition, improving overall well-being, increasing survival or a combination thereof.

44. The method of claim 42 or claim 43, wherein:
the peroxisome proliferator-activated receptor delta (PPARδ) agonist compound is administered to the mammal in an amount sufficient for increasing OXPHOS capacities in the mammal, up-regulating gene expression of any one of the enzymes or proteins involved in OXPHOS, or a combination thereof.

45. The method of any one of claim 43 or claim 44, wherein:
the peroxisome proliferator-activated receptor delta (PPARδ) agonist compound is administered to the mammal in an amount sufficient for increasing fatty acid oxidation (FAO) capacities in the mammal, up-regulating gene expression of any one of the enzymes or proteins involved in FAO, or a combination thereof.

46. The method of any one of claims 42-45, wherein the mammal with a primary mitochondrial myopathy has:
at least one mutation or deletion in at least one mitochondrial DNA (mtDNA) gene;
at least one mitochondrial DNA (mtDNA) defect;
at least one mutation or deletion in at least one nuclear DNA (nDNA) gene involved in mitochondrial function; or
a combination thereof.

47. The method of claim 46, wherein:
the at least one mutation in at least one mitochondrial DNA (mtDNA) gene comprises a mutation selected from m.3243A>G, m.8344A>G, m.8993T>G, m.13513G>A, m.11778G>A, m.14484T>C, and a combination thereof;
or the at least one mutation in at least one mitochondrial DNA (mtDNA) gene comprises a mutation selected from a 8284 bp deletion, a 6277 bp deletion, a 4977 bp deletion, and a combination thereof;
the at least one mutation or deletion in at least one nuclear DNA (nDNA) gene involved in mitochondrial function comprises at least one mutation or deletion in a nDNA gene encoding complex I (NADH:ubiquinone oxidoreductase), complex II (succinate dehydrogenase), complex III (CoQ-cytochrome c reductase), complex IV (cytochrome c oxidase), complex V (ATP synthase), an aminoacyl-tRNA synthetase, a release factor, an elongation factor, a mitoribosomal protein, solute carriers of thiamine and phosphate, or a combination thereof.

48. The method of claim 47, wherein:
the gene encoding the complex I comprises NDUFS1, NDUFS2, NDUFS3, NDUFS4, NDUFS6, NDUFS7, NDUFS8, NDUFVJ, NDUFV2, NDUFA1, NDUFA2, NDUFA9, NDUFA10, NDUFA11, NDUFA12, NDUFA13, NDUFAF2, NDUFAF6, or NDUFB11;
the gene encoding the complex II comprises SDHA, SDHB, SDHC, SDHD, or SDHAF1;
the gene encoding the complex III comprises UQCRB, BCS1L, UQCRQ, UQCRC2, CYC1, TTC19, LYRM7, UQCC2, or UQCC3;
the gene encoding the complex IV comprises COA5, SURF1, COX10, COX14, COX15, COX20, COX6B1, FASTKD2, SCO1, SCO2, LRPPRC, TACO1, or PET100;
the gene encoding the complex V comprises ATPAF2, TMEM70, ATP5E, or ATP5A1;
the gene encoding the aminoacyl-tRNA synthetase comprises AARS2, DARS2, EARS2, RARS2, YARS2, FARS2, HARS2, LARS2, VARS2, TARS2, IARS2, CARS2, PARS2, NARS2, KARS, GARS, SARS2, or M4RS2;
the gene encoding the release factor comprises C12orf65;
the gene encoding the elongation factor comprises TUFM, TSFM, or GFM1;
the gene encoding the mitoribosomal protein comprises MRPS16, MRPS22, MRPL3, MRP12, or MRPL44; and
the gene encoding the solute carriers of thiamine and phosphate comprises SLC19A3, SLC25A3, or SLC25A19.

49. The method of claim 46, wherein:
the at least one mutation or deletion in at least one nuclear DNA (nDNA) gene involved in mitochondrial function comprises at least one mutation or deletion in a nDNA gene involved in phospholipid metabolism, metabolism of toxic compounds, disulfide relay system, iron-sulfur protein assembly, tRNA modification, mRNA processing, mitochondrial fusion or fission, deoxynucleotide triphosphate synthesis, protein quality control and degradation, ATP and ADP transport, or a combination thereof.

50. The method of claim 49, wherein:
the gene involved in the phospholipid metabolism comprises AGK, SERAC1, or TAZ;
the gene involved in the metabolism of toxic compounds comprises HIBCH, ECHS1, ETHE1, or MPV17;
the gene involved in the disulfide relay system comprises GFER;
the gene involved in the iron-sulfur protein assembly comprises ISCU, BOLA3, NFU1, or IBA57;
the gene involved in the tRNA modification comprises MTO1, GTP3BP, TRMU, PUS1, MTFMT, TRIT1, TRNT1, or TRMT5;
the gene involved in the mRNA processing comprises LRPPRC, TACO1, ELAC2, PNPT1, HSD17B10, MTPAP, or PTCD1;
the gene involved in the mitochondrial fusion and fission comprises OPA1 or MFN2;
the gene involved in the deoxynucleotide triphosphate synthesis comprises DGUOK, TK2, TYMP, MGME1, SUCLG1, SUCLA2, RNASEH1, C10orf2, POLG, POLG2, DNA2, or RRM2B;
the gene involved in the protein quality control and degradation comprises FBXL4, AFG3L2, or SPG7; and
the gene involved the ATP and ADP transport comprises ANT1.

51. The method of any one of claims 42-50, wherein:
the mammal has been diagnosed with Kearns-Sayre syndrome (KSS), Leigh syndrome, maternally inherited Leigh syndrome (MILS), Mitochondrial DNA depletion syndrome (MDS), Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy ataxia and retinitis pigmentosa (NARP), Pearson syndrome, or Progressive external ophthalmoplegia (PEO).

52. The method of any one of claims 42-51, wherein:
the mammal with a primary mitochondrial myopathy also comprises a secondary mitochondrial myopathy.

53. The method of claim 52, wherein:
the secondary mitochondrial myopathy involves secondary defects in OXPHOS function due to primary FAO deficiencies, or the secondary mitochondrial myopathy results from a primary OXPHOS deficiency that results in secondary FAO disease.

54. The method of any one of claims 42-53, wherein the PPARδ agonist compound increases expression or activity of a gene or protein involved in mitochondrial biogenesis.

55. The method of claim 54, wherein the protein is peroxisome proliferator-activated receptor gamma coactivator 1-alpha (PGC-1α).

56. The method of any one of claims 42-55, wherein, the PPARδ agonist compound increases expression or activity of a gene or protein thereof involved in oxidative phosphorylation.

57. The method of any one of claims 42-56, wherein, the PPARδ agonist compound increases the percentage of non-mutated mitochondrial DNA (mtDNA) relative to the proportion of mutated mtDNA.

58. The method of any one of claims 42-57, wherein:
(E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg.

59. The method of any one of claims 42-57, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg.

60. The method of any one of claims 42-57, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 75 mg to about 125 mg.

61. The method of any one of claims 42-57, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg.

62. The method of any one of claims 42-57, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 100 mg.

63. The method of any one of claims 42-62, wherein:
(E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, is systemically administered to the mammal in the form an oral solution, oral suspension, powder, pill, tablet or capsule.

64. The method of any one of claims 42-63, wherein:
the PPARδ agonist compound is administered to the mammal daily.

65. The method of any one of claims 42-63, wherein:
the PPARδ agonist compound is administered to the mammal once daily.

66. The method of any one of claims 42-65, further comprising:
administering at least one additional therapeutic to the mammal.

67. The method of claim 66, wherein:
the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, KH176, or a combination thereof.

68. The method of claim 66, wherein:
the at least one additional therapeutic is an odd-chain fatty acid, odd-chain fatty ketone, L-carnitine, or combinations thereof.

69. The method of claim 66, wherein:
the at least one additional therapeutic is triheptanoin, n-heptanoic acid, a triglyceride, or a salt or thereof, or combinations thereof.

70. The method of any one of claims 42-69, wherein the mammal is a human.

71. A method for treating a primary mitochondrial myopathy in a human comprising administering to the mammal with a primary mitochondrial myopathy a PPARδ agonist compound, wherein after treatment the mammal has improvement in one or more of pain, cognition, physical endurance, muscle strength, feeling of well-being, or increasing survival.

72. The method of claim 71, wherein the improvement is physical endurance.

73. The method of claim 72, wherein the improvement is physical endurance as demonstrated by one or more of improvement in walking endurance, or sit to stand test.

74. The method of claim 71, wherein the improvement is muscle strength.

75. The method of claim 74, wherein the muscle strength is measured by grip strength, or leg strength.

76. The method of claim 71, wherein the improvement is increasing survival of the human.

77. The method of any one of claims 71-76, wherein the PPARδ agonist compound is:
(E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg.

78. The method of claim 77, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 50 mg to about 200 mg.

79. The method of any one of claim 77, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 75 mg to about 125 mg.

80. The method of any one of claim 77, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 50 mg.

81. The method of claim 77, wherein:
(E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof is administered to the mammal at a dose of about 100 mg.

82. The method of any one of claims 77-81, wherein:
(E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof is systemically administered to the mammal in the form an oral solution, oral suspension, powder, pill, tablet or capsule.

83. The method of claims 71-82, wherein the PPARδ agonist compound is administered to the mammal daily.

84. The method of claim 83, wherein the PPARδ agonist compound is administered to the mammal once daily.

85. The method of any one of claims 71-84, further comprising administering at least one additional therapeutic.

86. The method of claim 85, wherein the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, acipimox, elamipretide, cysteamine, succinate, NAD agonists, vatiquinone (EPI-743), omaveloxolone (RTA-408), nicotinic acid, nicotinamide, elamipretide, KL133, KH176, or a combination thereof.

87. The method of claim 85, wherein the at least one additional therapeutic is an odd-chain fatty acid, odd-chain fatty ketone, L-carnitine, or combinations thereof.

88. The method of claim 85, wherein the at least one additional therapeutic is triheptanoin, n-heptanoic acid, a triglyceride, or a salt or thereof, or combinations thereof.
